(19) United States
(12) Patent Application Publication
(10) Pub. No.: US 2023/0233089 A1
(43) Pub. Date: Jul. 27, 2023

(54) MULTI-SENSOR MEMS SYSTEM AND MACHINE-LEARNED ANALYSIS METHOD FOR HYPERTROPHIC CARDIOMYOPATHY ESTIMATION

(71) Applicant: Analytics for Life Inc., Toronto (CA)

(72) Inventors: Charles R. Bridges, Auburndale, MA (US); Farhad Fathieh, North York (CA); Shyamlal Ramchandani, Kingston (CA); Jonathan James Woodward, Annapolis, MD (US)

(21) Appl. No.: 18/158,111

(22) Filed: Jan. 23, 2023

Related U.S. Application Data
(60) Provisional application No. 63/302,109, filed on Jan. 23, 2022.

(51) Int. Cl. A61B 5/02; G16H 50/20

(52) U.S. Cl. CPC A61B 5/02; G16H 50/20; A61B 5/72

(57) ABSTRACT

An exemplary method is disclosed that can be used in the diagnosis of hypertrophic cardiomyopathy (HCM) using a biophysical-sensor system configured to non-invasively and concurrently acquire electrocardiographic signals, seismographic signals, photoplethysmographic, and/or phonocardiographic signals, collectively referred to herein as biophysical signals, from at least the thoracic region of a subject. The acquired biophysical signals may be assessed for one or more conditions or indicators of hypertrophic cardiomyopathy and concurrently with other cardiac diseases, conditions, or indicators of either.

### RELATED APPLICATION

This application claims priority to, and the benefit of, U.S. Provisional Patent Application No. 63/302,109, filed Jan. 23, 2022, entitled “MEMS-SENSOR SYSTEM AND MACHINE-LEARNED ANALYSIS METHOD FOR HYPERTROPHIC CARDIOMYOPATHY ESTIMATION,” which is hereby incorporated by reference herein in its entirety.

### BACKGROUND

Hypertrophic cardiomyopathy (HCM) is a disease, typically of an autosomal dominant genetic origin, that causes myocytes in the heart wall (cardiomyocytes) to increase in size and cause one or more regions of the heart wall to become abnormally thick (hypertrophied). It may result in diastolic and/or systolic dysfunction with clinical manifestations of heart failure or valvular heart disease. While there are about 100,000 people diagnosed with HCM in the United States, it is expected that over 500,000 people in the United States may have this condition [16]. HCM can cause sudden cardiac death in about 1% of the affected population per year. Its symptoms include, e.g., chest pain, fainting (syncope), a sensation of heart palpitation, and shortness of breath. Current diagnostic regimens for HCM include genetic testing, echocardiogram, electrocardiogram (ECG/EKG), and cardiac magnetic resonance imaging (MRI). Some of these tests can be costly and often require specialized equipment in a dedicated room in a hospital or clinical center and experienced technicians to perform them. They often are separately scheduled to be performed days or weeks after an initial visit with a healthcare provider.

There is interest in the early diagnosis and treatment of HCM, for example, to reduce or prevent the cardiomyocytes from further enlarging into a hypertrophied state. Such early diagnosis and/or treatment can lead to improved health and overall outcomes for patients having this condition. Early diagnosis and treatment of HCM can save lives and save healthcare costs as more costly interventions and therapies can be avoided or mitigated.

There are also benefits in being able to systematically screen or evaluate for the presence, non-presence, and/or severity of HCM—no matter what stage of the disease—using non-invasive techniques without the use of radiation, drugs, and/or stress, and do so more quickly and cost-effectively than present methods allows so that assessing (e.g., predict and/or detect) the presence, non-presence, severity and (in some cases) localization of various diseases, pathologies or conditions in mammalian or non-mammalian organisms may be accomplished more safely, with lower costs, and/or in a shorter amount of time than current methods and systems provide.

The methods and systems described herein address this need and may be used for a wide variety of clinical and even research needs in a wide variety of settings—from hospitals to emergency rooms, laboratories, battlefields, remote settings, at point of care with a patient's primary care physician or other caregivers, and even the home.

### SUMMARY

An exemplary method is disclosed that can be used in the diagnosis of hypertrophic cardiomyopathy (HCM) using a biophysical-sensor system (e.g., a multi-sensor system) configured to non-invasively and concurrently acquire electrocardiographic signals, seismographic signals, photoplethysmographic, and/or phonocardiographic signals, among others (collectively referred to herein as biophysical signals), from a subject. One or more of these signals may be collected from the thoracic region of a patient. The acquired biophysical signals may be assessed for one or more conditions or indicators of hypertrophic cardiomyopathy and concurrently with other diseases, conditions, or indicators of either. The biophysical-sensor system may include MEMS-based accelerometers, transducers, or sensors to acquire the seismocardiographic signals and/or phonocardiographic signals as well as other related signals such as ballistocardiographic signals. The biophysical-sensor system may include surface electrode-based acquisition circuitry or module to directly acquire electrocardiographic signals or cardiac signals. The biophysical-sensor system may include a photoplethysmographic sensor or module to directly acquire photoplethysmographic signals or other hemodynamic signals. The biophysical-sensor system may employ wire or wireless communication or may be an independent or integrated sensor. The biophysical-sensor system may operatively connect and operate as part of a clinical evaluation system that includes an analysis system comprising an analytical engine configured to perform machine-learned-based analysis to provide one or more estimated metrics associated with the presence, non-presence, and/or severity of hypertrophic cardiomyopathy or conditions (such as described herein) that might otherwise not be detectable or understandable to the same degree. The clinical evaluation system, in some embodiments, may include additional analytical engines configured to perform additional machine-learned-based analyses to assess for other physiological states of a patient, including for the presence or non-presence of other diseases, medical conditions, or indications of either, including disease and conditions such as (i) heart failure (e.g., left-side or right-side heart failure; heart failure with preserved ejection fraction (HFpEF), heart failure with reduced ejection fraction (HFrEF)), (ii) coronary artery disease (CAD), (iii) various forms of pulmonary hypertension (PH) including, and without limitation, pulmonary arterial hypertension (PAH), (iv) abnormal left ventricular ejection fraction (LVEF), and various other diseases or conditions. Examples of indicators of certain forms of disease that can be evaluated include the presence or non-presence of elevated or abnormal left-ventricular end-diastolic pressure (LVEDP) as an indication of heart failure or elevated or abnormal mean pulmonary arterial pressure (mPAP) as an indication of pulmonary hypertension.

In an aspect, a method is disclosed to non-invasively estimate a presence, non-presence, and/or severity of hypertrophic cardiomyopathy in a mammalian subject, the method comprising obtaining, by one or more processors, one or more biophysical signals of a patient from one or more sensors; determining, by the one or more processors utilizing at least a portion of the one or more signals, one or more values associated with one or more features and/or machine-learned-based analyses; and determining, by the one or more processors, an estimated value for the presence, non-presence, and/or severity of hypertrophic cardiomyopathy using the plurality of values associated with the plurality of features or machine-learned-based analyses (e.g., in a trained machine learning or AI model), wherein the estimated value (e.g., an HCM score) for the presence, non-presence, and/or severity of hypertrophic cardiomyopathy is outputted for use in a diagnosis of hypertrophic cardiomyopathy and/or to direct treatment of the hypertrophic cardiomyopathy. In some embodiments, the method further includes outputting the estimated value for that usage.

In another aspect, a method is disclosed to non-invasively estimate a presence, non-presence, and/or severity of hypertrophic cardiomyopathy in a mammalian subject, the method comprising obtaining, by one or more processors, one or more seismocardiographic signals (SCG signals) and/or phonocardiographic signals (PCG signals) from a multi-sensor device placed or worn on a patient; determining, by the one or more processors utilizing at least a portion of the one or more seismocardiographic and/or phonocardiographic signals, a plurality of values associated with a plurality of features or machine-learned-based analyses; and determining, by the one or more processors, an estimated value for the presence, non-presence, and/or severity of hypertrophic cardiomyopathy using the plurality of values associated with the plurality of features or machine-learned-based analyses (e.g., in a trained machine learning or AI model), wherein the estimated value (e.g., an HCM score) for the presence, non-presence, and/or severity of hypertrophic cardiomyopathy is outputted for use in a diagnosis of hypertrophic cardiomyopathy or to direct treatment of the hypertrophic cardiomyopathy. In some embodiments, the method further includes outputting the estimated value for that usage.

In another aspect, a method is disclosed to non-invasively estimate a presence, non-presence, and/or severity of hypertrophic cardiomyopathy in a mammalian subject, the method comprising obtaining, by one or more processors, a first biophysical signal data set (PPG signals) associated with a first photoplethysmographic signal and a second photoplethysmographic signal, wherein the first biophysical data set has been acquired over multiple cardiac cycles of the subject; obtaining, by the one or more processors, a second biophysical signal data set (cardiac signals) associated with a cardiac signal, wherein the second biophysical data set has been simultaneously acquired with the first biophysical signal data set over the multiple cardiac cycles; determining, by the one or more processors utilizing at least a portion of the first biophysical signal and the second biophysical signal, a plurality of values associated with a plurality of features or machine-learned-based analyses; and determining, by the one or more processors, an estimated value for the presence, non-presence, and/or severity of hypertrophic cardiomyopathy using the plurality of values associated with the plurality of features or machine-learned-based analyses (e.g., in a trained machine learning or AI model), wherein the estimated value (e.g., an HCM score) for the presence, non-presence, and/or severity of hypertrophic cardiomyopathy is outputted for use in a diagnosis of hypertrophic cardiomyopathy or to direct treatment of the hypertrophic cardiomyopathy. In some embodiments, the method further includes outputting the estimated value for that usage.

In some embodiments, the plurality of features or machine-learned-based analyses are configured to quantify deviations of a VD wave trajectory from a trajectory of the three-dimensional-modeled VD wave (e.g., to evaluate the high-frequency & low-amplitude patterns in the VD trajectory).

In some embodiments, the plurality of features or machine-learned-based analyses are configured to quantify the beat-to-beat variations in cardiac signals.

In some embodiments, the plurality of features or machine-learned-based analyses are configured to quantify variability in registered landmarks in cardiac, PPG, and/or SCG signals via Poincare analysis and histogram analysis.

In some embodiments, the plurality of features or machine-learned-based analyses are configured to quantify dynamical characteristics of cardiac, PPG, and/or SCG signals (e.g., Lyapunov exponent, correlation dimension, entropy, mutual information, correlation, and/or nonlinear filtering).

In some embodiments, the plurality of features or machine-learned-based analyses are configured to quantify properties of cardiac, PPG, and/or SCG signals (e.g., waveform amplitudes, durations, heart rate, morphologies; properties of PPG, VPG, APG signals, e.g., as peak amplitudes, peak-to-peak distances, angles between points, and various ratios).

In some embodiments, the plurality of features or machine-learned-based analyses are configured to quantify the main frequency components of cardiac, PPG, and/or SCG signals using wavelet analysis.

In some embodiments, the plurality of features or machine-learned-based analyses are configured to quantify power spectrum and frequency contents of the cardiac, PPG, and/or SCG signals) using power spectrum and coherence (cross-spectral analysis) analysis.

In some embodiments, the plurality of features or machine-learned-based analyses are configured to quantify properties of cardiac, PPG, and/or SCG signals over loop regions (e.g., atrial depolarization, ventricular depolarization, and ventricular repolarization) in 3D phase spaces, projections thereof, and loop vectors.

In some embodiments, the plurality of features or machine-learned-based analyses are configured to approximate a respiration waveform using either (i) PPG and cardiac signals or (ii) SCG signals to assess (1) heart rate variability, (2) respiration rate, (3) discrepancy features representing the distance between respiration and modulation signals and (4) square coherence, representing the correlation between modulation and respiration rate signals, wherein the approximated respiration waveform is employed for HCM assessment by being used to generate delineated inspiration and expiration portions of the SCG signals to be employed for the analysis.

In some embodiments, the plurality of features or machine-learned-based analyses are configured to quantify physiological aspects of the cardiac and/or SCG signals.

In some embodiments, the plurality of features or machine-learned-based analyses are configured to quantify characteristic variations in cardiac, SCG, and/or PCG signals associated with inspiration versus expiration versus the Valsalva maneuver to identify patients with HCM.

In some embodiments, the plurality of features or machine-learned-based analyses are configured to quantify characteristic variations in cardiac, SCG, and/or PCG signals associated with inspiration versus expiration versus the Valsalva maneuver to identify the subset of patients with HCM that have obstructive HCM (HOCM).

In some embodiments, the plurality of features or machine-learned-based analyses are configured to approximate left ventricular ejection time (LVET) using the one or more SCG and/or PCG signals.

In some embodiments, the plurality of features or machine-learned-based analyses are configured to quantify propagative characteristics of the ventricular depolarization (VD) wave and/or ventricular repolarization (VR) wave in three-dimensional space (e.g., velocity, trajectory, orbital frequency, planarity of the wave).

In some embodiments, the plurality of features or machine-learned-based analyses are evaluated (i) at an inspiration region of the one or more seismocardiographic signals and/or phonocardiographic signals and/or (ii) an expiration region of the one or more seismocardiographic signals and/or phonocardiographic signals.

In another aspect, an apparatus (e.g., SCG/PCG measurement device) is disclosed, comprising a sensor body configured to be externally worn or placed on a chest region of a subject to acquire biophysical signals from the chest region, including signals of a heart; and two or more MEMS-based biophysical sensors (e.g., accelerometers: single axis, or multi-axis), including a first MEMS-based sensor and a second MEMS-based sensor, wherein the two or more MEMS-based sensors are located within the sensor body and connected to an electrode configured to be placed on a subject, wherein the first MEMS-based sensor and the second MEMS-based sensor during operation generate a first biophysical signal and a second biophysical signal to be provided to an analysis system configured to evaluate the plurality of features or machine-learned-based analyses to generate an estimated value (e.g., an HCM score) for a presence, non-presence, and/or severity of hypertrophic cardiomyopathy (e.g., for use in a diagnosis of hypertrophic cardiomyopathy or to direct treatment of the hypertrophic cardiomyopathy) (e.g., in a trained machine learning or AI model).

In another aspect, an apparatus (e.g., SCG/PCG measurement device) is disclosed comprising a sensor body configured to be externally worn or placed on a chest region of a subject to acquire biophysical signals from the chest region, including of a heart; and two or more MEMS-based accelerometers (single axis, or multi-axis), including a first MEMS-based accelerometer and a second MEMS-based accelerometer, wherein the two or more MEMS-based accelerometers are located within the sensor body and connected to an electrode configured to be placed on a subject, wherein the first MEMS-based accelerometer and the second MEMS-based accelerometer during operation generate a first seismographic signal and a second seismographic signal to be provided to an analysis system configured to evaluate the plurality of features or machine-learned-based analyses to generate an estimated value (e.g., an HCM score) for a presence, non-presence, and/or severity of hypertrophic cardiomyopathy (e.g., for use in a diagnosis of hypertrophic cardiomyopathy or to direct treatment of the hypertrophic cardiomyopathy) (e.g., in a trained machine learning or AI model).

In some embodiments, the apparatus further includes a plurality of surface electrodes configured to be placed on surfaces of a chest region of a subject to provide a plurality of cardiac signals of the subject's heart, wherein the plurality of cardiac signals are provided to the analysis system to evaluate for the plurality of features or machine-learned-based analyses to generate the estimated value for the presence, non-presence, and/or severity of hypertrophic cardiomyopathy.

In some embodiments, the apparatus further includes a plurality of photoplethysmographic sensors configured to be placed on the subject to provide one or more photoplethysmographic signals, wherein the one or more photoplethysmographic signals are provided to the analysis system to evaluate for the plurality of features or machine-learned-based analyses to generate the estimated value for the presence, non-presence, and/or severity of hypertrophic cardiomyopathy.

In some embodiments, the first MEMS-based accelerometer is configured to be placed adjacent to an apex region of the heart.

In some embodiments, the second MEMS-based accelerometer is configured to be placed adjacent to a base region of the heart.

In another aspect, a system is disclosed comprising any of the above-discussed apparatus and the analysis system, wherein the analysis system is implemented in a cloud-based processing and networking infrastructure.

In another aspect, a system (e.g., cloud platform or local computing platform) is disclosed comprising one or more processors and one or more memory having instructions respectively stored thereon, wherein execution of the instructions by the one or more processors causes the one or more processors to perform any one of the above-discussed methods.

In another aspect, a non-transitory computer-readable medium is disclosed comprising instructions stored thereon, wherein execution of the instructions by one or more processors causes the one or more processors to perform any one of the above-discussed methods.

In another aspect, a system is disclosed comprising an apparatus comprising: a sensor body configured to be externally worn or placed on a chest region of a subject to acquire biophysical signals from the subject's chest region, including signals of the subject's heart; and two or more MEMS-based biophysical sensors, including a first MEMS-based sensor and a second MEMS-based sensor, wherein the two or more MEMS-based sensors are located within the sensor body and connected to an electrode configured to be placed on a subject, wherein the first MEMS-based sensor and the second MEMS-based sensor during operation generate a first biophysical signal and a second biophysical signal to be provided to an analysis system configured to evaluate the plurality of features or machine-learned-based analyses to generate an estimated value for a presence, non-presence, and/or severity of hypertrophic cardiomyopathy (e.g., in a trained machine learning or AI model); and the analysis system, wherein the analysis system is implemented in a cloud-based processing and networking infrastructure.

In another aspect, a non-transitory computer-readable medium comprising instructions stored thereon, wherein execution of the instructions by one or more processors causes the one or more processors to: obtain one or more seismocardiographic signals (SCG signals) and/or phonocardiographic signals (PCG signals) from a multi-sensor device placed or worn on a patient; determine utilizing at least a portion of the one or more seismocardiographic signals and/or phonocardiographic signals, a plurality of values associated with a plurality of features or machine-learned-based analyses; determine an estimated value for the presence, non-presence and/or severity of hypertrophic cardiomyopathy using the plurality of values associated with the plurality of features or machine-learned-based analyses (e.g., in a trained machine learning or AI model); and output the estimated value for the presence, non-presence, and/or severity of hypertrophic cardiomyopathy, wherein the estimated value for the presence, non-presence, and/or severity of hypertrophic cardiomyopathy is outputted for use in a diagnosis of hypertrophic cardiomyopathy and/or to direct treatment of the hypertrophic cardiomyopathy.

In another aspect, a non-transitory computer-readable medium comprising instructions stored thereon, wherein execution of the instructions by one or more processors causes the one or more processors to: obtain one or more biophysical signals of a patient from one or more sensors; determine uilizing at least a portion of the one or more signals, one or more values associated with one or more features and/or machine-learned-based analyses; determine an estimated value for the presence, non-presence, and/or severity of hypertrophic cardiomyopathy using the plurality of values associated with the plurality of features or machine-learned-based analyses (e.g., in a trained machine learning or AI model); and output the estimated value for the presence, non-presence, and/or severity of hypertrophic cardiomyopathy, wherein the estimated value for the presence, non-presence, and/or severity of hypertrophic cardiomyopathy is outputted for use in a diagnosis of hypertrophic cardiomyopathy and/or to direct treatment of the hypertrophic cardiomyopathy.

In another aspect, a non-transitory computer-readable medium is disclosed comprising instructions stored thereon, wherein execution of the instructions by one or more processors causes the one or more processorss: obtain a first biophysical signal data set associated with a first photoplethysmographic signal and a second photoplethysmographic signal, wherein the first biophysical data set has been acquired over multiple cardiac cycles of the subject; obtain a second biophysical signal data set associated with a cardiac signal, wherein the second biophysical data set has been simultaneously acquired with the first biophysical signal data set over the multiple cardiac cycles; determine utilizing at least a portion of the first biophysical signal and the second biophysical signal, a plurality of values associated with a plurality of features or machine-learned-based analyses; determine an estimated value for the presence, non-presence, and/or severity of hypertrophic cardiomyopathy using the plurality of values associated with the plurality of features or machine-learned-based analyses (e.g., in a trained machine learning or AI model); and output the estimated value for the presence, non-presence, and/or severity of hypertrophic cardiomyopathy, wherein the estimated value for the presence, non-presence, and/or severity of hypertrophic cardiomyopathy is outputted for use in a diagnosis of hypertrophic cardiomyopathy and/or to direct treatment of the hypertrophic cardiomyopathy.

### BRIEF DESCRIPTION OF THE DRAWINGS

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a schematic diagram of a clinical evaluation system configured to non-invasively and concurrently acquire electrocardiographic signals, seismographic signals, photoplethysmographic, and/or phonocardiographic signals and to evaluate the acquired biophysical signals in machine-learned-based analysis to generate one or more estimated metrics associated with the presence, non-presence, and/or severity of hypertrophic cardiomyopathy.

FIG. 2 shows a diagram of example HCM physiological effects and example machine-learned-based analyses.

FIG. 3 shows a diagram of example biophysical signals acquired for the evaluation of hypertrophic cardiomyopathy in accordance with an illustrative embodiment.

FIG. 4A shows a diagram of an example SCG/PCG measurement device in accordance with an illustrative embodiment.

FIG. 4B shows a diagram of another example SCG/PCG measurement device in accordance with an illustrative embodiment.

FIG. 4C shows a diagram of yet another example SCG/PCG measurement device in accordance with an illustrative embodiment.

FIG. 4D is a diagram of an example measurement system configured as a wearable MEMS sensor device in accordance with an illustrative embodiment.

FIG. 4E shows images of the example fabricated wearable MEMS sensor device of FIG. 4D, configured as a wearable chest sensor device in accordance with an illustrative embodiment.

FIG. 5A shows a schematic diagram of an example clinical evaluation system configured to use machine-learned-based analyses (among other analyses) to generate one or more metrics associated with the physiological state of a patient, including, e.g., presence, non-presence, and/or severity of HCM, another condition, or indications and/or severity of either, in accordance with an illustrative embodiment.

FIG. 5B shows a schematic diagram of the operation of the example clinical evaluation system of FIG. 5A in accordance with an illustrative embodiment.

FIGS. 6A-6H show experimental results of various developed ML features to determine HCM physiological effects in accordance with an illustrative embodiment.

### DETAILED DESCRIPTION

Each and every feature described herein, and each and every combination of two or more of such features, is included within the scope of the present invention provided that the features included in such a combination are not mutually inconsistent.

### Definitions

The terms “subject” and “patient” as used herein are generally used interchangeably to refer to those who had undergone analysis performed by the exemplary systems and methods.

The term “biophysical signal” as used herein includes but is not limited to one or more cardiac signal(s), neurological signal(s), seismocardiographic signals, ballistocardiographic signal(s), and/or photoplethysmographic signal(s), phonocardiographic signal(s), and/or seismocardiographic signal(s) but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may, in some cases, be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “cardiac signal” as used herein refers to one or more signals directly or indirectly associated with the structure, function, and/or activity of the cardiovascular system—including aspects of that signal's electrical/electrochemical conduction—that, e.g., cause contraction of the myocardium. A cardiac signal may include, in some embodiments, biopotential signals or electrocardiographic signals, e.g., those acquired via an electrocardiogram (ECG), the cardiac and photoplethysmographic waveform or signal capture or recording instrument later described herein, or other modalities. Cardiac signals, in some embodiments, are acquired as orthogonal voltage gradient (OVG) signals.

The term “photoplethysmographic signal,” as used herein, refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation, e.g., using an accelerometer such as an electro-mechanical-system based (MEMS) accelerometer or transducer. In other embodiments, ballistocardiographic signals may be acquired by external equipment, e.g., a bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

The term “seismocardiogram signal,” as used herein, refers to a signal or group of signals that generally reflect recorded body's vibrations, sound, or orientation as recorded by sensors, e.g., MEMS sensors such as a MEMS accelerometer, mounted or positioned close to the heart. The term “seismocardiogram signal” is interchangeably used with the term “seismocardiographic signal.”

The term “phonocardiogram signal,” as used herein, refers to a signal or group of signals that generally reflect recorded body's sound, vibrations, and acoustic radiation as recorded by sensors, e.g., microphones or accelerometers mounted or positioned close to the heart or around the thoracic region of a subject. The term “phonocardiogram signal” is interchangeably used with the term “phonocardiographic signal.”

Example System

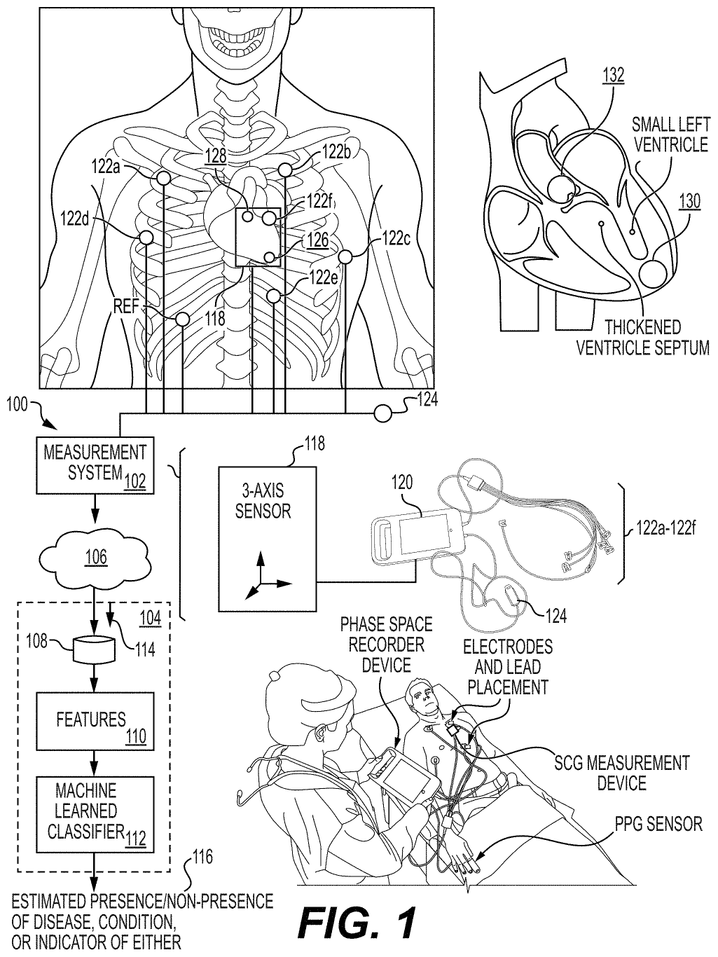

FIG. 1 is a schematic diagram of a clinical evaluation system 100 configured to non-invasively and concurrently acquire electrocardiographic (cardiac) signals, one or more seismocardiographic (SCG) signals, (PPG) photoplethysmographic, and/or phonocardiographic (PCG) signals and to evaluate the acquired biophysical signals in machine-learned-based analysis to generate one or more estimated metrics associated with the presence, non-presence, and/or severity of hypertrophic cardiomyopathy. System 100 includes a measurement system 102 and an analysis system 104 over a network 106. In the example shown in FIG. 1, the analysis system 104 is implemented in a cloud-based infrastructure. Analysis system 104 includes (i) a data store 108 to receive one or more data files 114 associated with a measurement performed by the measurement system 102 and (ii) an analytical engine comprising analytics feature analysis module 110 (shown as “Features” 110) and a machine-learned classifier module 112 to evaluate the one or more data files 114 in the machine-learned-based analysis. The output 116 of the analysis system 104 may be provided as a patient's report, e.g., via a healthcare portal or as output to a wearable device, or as output to a piece of medical equipment for the treatment of a disease, condition, or indication of either. A healthcare provider, e.g., a physician, can review the report and interpret it to provide a diagnosis of the disease or to generate a treatment plan.

Machine-learned-based analysis refers to analyses or features that include, or are derived from, a machine learning or artificial intelligence analysis. The machine-learned-based analysis, in some embodiments, includes the evaluation of features from a library of features to down-select or train with clinical data to features that are statistically significant in the estimation of metrics associated with the presence, non-presence, and/or severity of hypertrophic cardiomyopathy, e.g., via ElasticNet machine-learned classifier models [9], RandomForestClassifier machine-learned classifier models [10], and extreme gradients boosting (XGB) classifier models [11]. Examples of training systems to configure the analysis system (e.g., for elevated LVEDP estimation) are described in U.S. Provisional Application No. 63/235,960, filed Aug. 23, 2021, entitled “METHOD AND SYSTEM TO ASSESS HEART FAILURE,” which is hereby incorporated by reference herein in its entirety. Another example of a training system that may be used to configure the analytical engine is described in [19], which is hereby incorporated by reference herein in its entirety.

HCM Physiological Effects and Indicators

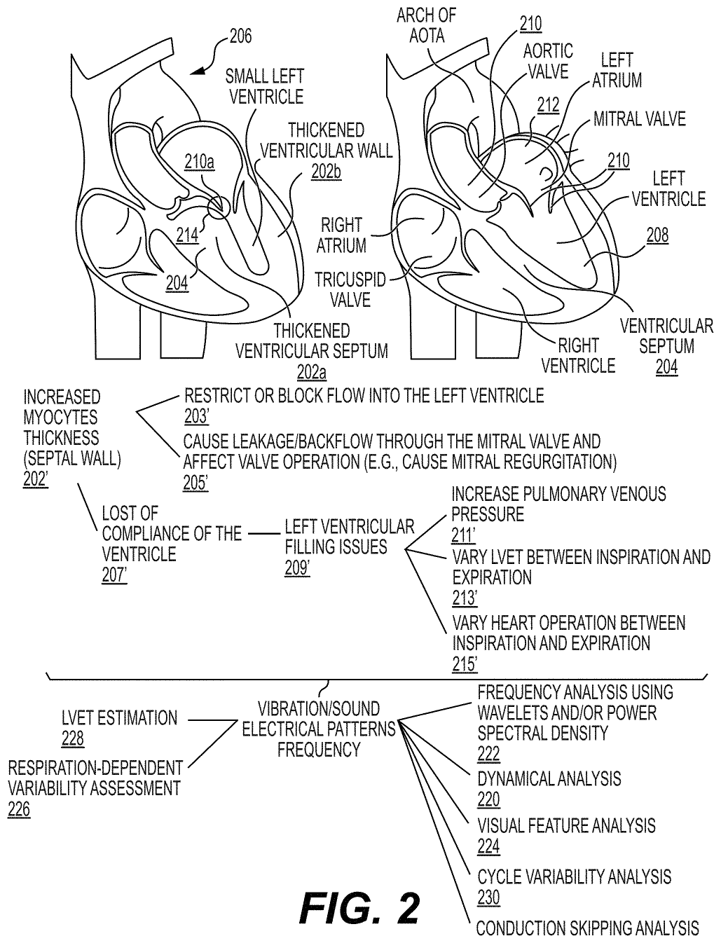

FIG. 2 shows a diagram of example HCM physiological effects and example machine-learned-based analyses. Hypertrophic cardiomyopathy (HCM) is a genetic disorder that is characterized by left ventricular hypertrophy unexplained by secondary causes and a non-dilated left ventricle, typically with preserved or increased ejection fraction. It is commonly asymmetric, with the most severe hypertrophy involving the basal interventricular septum. Left ventricular outflow tract obstruction is present at rest in about one-third of the patients and can be provoked in another third [21]. The pathological hallmarks of the disease include myocyte hypertrophy and disarray, interstitial and replacement fibrosis, small vessel abnormalities, and electrical remodeling of the cardiomyocyte, which may form a substrate for ventricular arrhythmias. Hypertrophy is also frequently associated with left ventricular diastolic dysfunction [22]. In addition, a high prevalence of abnormal sinus-node function (66%) and His-Purkinje (HV) conduction (30%) was observed in HCM patients with electrophysiologic abnormalities [23]. The most commonly induced supraventricular arrhythmias can be atrial reentrant tachycardia and atrial fibrillation (10% and 11% of patients, respectively).

In HCM patients, the heart wall(s) (e.g., 202a, 202b) are enlarged due to the cardiomyocytes being enlarged (hypertrophic), resulting in an increase in the cardiac wall size, mass, and stiffness (or loss of compliance). HCM hearts can exhibit different topologies. In some instances, the thickening can be asymmetric, e.g., involving only the septum between the left and right ventricles (202a), or the circumferential thickness of the left ventricular wall (202b) can be increased concentrically.

Enlargement effects (202′). When the septal wall (204) (shown as ventricular septum 204) is enlarged that it infringes on the left ventricular outflow tract (see 206); it can create a physiological scenario similar to an aortic valve blockage that blocks the blood flow out of the left ventricle (208) into the aorta (201). This blockage or impediment of flow (203′) has been observed as turbulent flow through the narrowed left ventricular outflow tract (LVOT), causing “murmurs” (obstruction murmurs) or sounds and vibrations similar to all of the physiological effects of aortic valve stenosis (narrowing of the exit of the left ventricle of the heart).

In addition to this flow or impediment blockage (203′), the infringement by the septal wall (204) into the left ventricular chamber (208) can also affect the functioning of the mitral valve (210), which can cause mitral regurgitation (a condition in which the mitral valve, comprising two cusps or flaps and is located between the left atrium (212) and the left ventricle (208) of the heart, does not close tightly, allowing blood to flow backward in the left atrium (212)) (205′). As a result of this systolic anterior motion (SAM) of the mitral valve (210), the anterior mitral leaflet can be pulled toward (214) the septum, making the mitral valve (210a) incompetent. The leakage (205′) across the mitral valve (e.g., 210a) can cause enlargement of the heart and clinical manifestations of heart failure, as can aortic stenosis. Mitral regurgitation (205′), with associated leakage across the mitral valve, often causes turbulent flow, heart murmurs (“mitral regurgitation murmur”), and vibrations.

Altered Tissue Stiffness (207′). Because the ventricle muscle (202b) is thicker, it is also stiffer and less dispensable, it may have physiology similar to heart failure with preserved ejection fraction (HFpEF) in which the heart can contract perfectly well, but it can not relax properly. HfpEF is characterized by abnormal diastolic function in which an increase in the stiffness of the left ventricle walls (202b) causes a decrease in the left ventricular relaxation during diastole, leading to increased pressure and/or impaired filling. In other cases, patients with HCM have both impaired diastolic function and a reduction in systolic function as well. All of these effects (loss of compliance of the heart tissue (207′), blockages/flow impediment (203′), valve leakages (205′)), impaired systolic and/or diastolic function and can cause pulmonary congestion, leading to shortness of breath and syncope (temporary loss of consciousness usually related to insufficient blood flow to the brain), among other symptoms.

The decrease in the left ventricular relaxation during diastole, in essence, is a filling difficulty (209′) of the left ventricle (208) (and not a difficulty of the ventricle contracting). It can be modeled or considered as a muscle-bounded heart that is hindered in its ability sufficiently to relax to allow blood to flow in and, thus, can lead to elevated left atrial pressure (represents the pulmonary venous pressure), elevated left ventricular pressure (LVP) (pressure in the left ventricle), and elevated diastolic pressure (pressure the blood is exerting against the artery walls when the heart is relaxed between beats). To get an adequate filling, the heart can increase the pulmonary venous pressure (211′), which can cause congestion (fluid buildup in the lungs) and shortness of breath, among other effects. The effect of the heart muscle being able to squeeze adequately but not relax adequately to fill for the next heartbeat can also lead to the body compensating by increasing arterial and venous blood pressure; this can also lead to shortness of breath and other conditions.

All of these effects (loss of compliance heart tissue, blockages/flow impediment, valve leakages, loss of compliance of the heart tissue) can affect the voltage distribution and pattern of the heart as compared to a normal heart. By virtue of the change in the heart's geometry (asymmetric or symmetric), heart operation can be altered in time and indicated by changes in the left ventricular ejection time (LVET) (213′). LVET measures the period of blood flow across the aortic valve and has a normal value of 0.35±0.08 seconds.

In addition, all of these effects (loss of compliance of the heart tissue, blockages/flow impediment, valve leakages) can affect frequent observable components of the heart. For example, the frequency components (e.g., as observed through power-spectral density or wavelet frequency components, among others) of the heart may vary with respiratory frequency. During inspiration, there is more filling of the heart as compared to expiration because the transthoracic pressure during inspiration can augment the ventricular filling pressure. The transthoracic pressure can be viewed as a hemodynamic pressure combined with negative intrathoracic pressure from inspiration.

In addition, there may be observable phasic elements within the sound due to the loss of compliance of the heart tissue, blockages/flow impediments, and valve leakages. With deep inspiration, the heart can have more filling and less obstruction of the left ventricular outflow, while, with expiration, there can be less filling and more obstruction of the outflow tract [17]. Heart murmurs can potentially be heard with decreased amplitude during inspiration and increased amplitude with expiration. Similarly, vibrations detected as seismocardiographic and/or phonocardiographic signals may decrease in amplitude during inspiration and increase in amplitude with expiration. Similarly, vibrations detected as seismocardiographic and/or phonocardiographic signals may increase in amplitude with a physiological maneuver known as the Valsalva maneuver. The Valsalva maneuver is a breathing protocol that can be performed by a forceful attempt of exhalation against a closed airway, usually done by closing one's mouth and pinching one's nose shut while expelling air out as if blowing up a balloon.

Indeed, as noted above, the above-discussed body physiology and HCM physiological effects can generate vibration, electrical patterns, and frequency components that are indicative or unique to HCM and its onset conditions. These physical manifestations can be evaluated by features or machine-learned-based analysis described herein to estimate metrics associated with the presence, non-presence, and/or severity of hypertrophic cardiomyopathy.

In addition, the presence of ventricular tachyarrhythmias can be an indication of the presence of hypertrophic cardiomyopathy. Ventricular tachycardia (V-tach or VT) may be characterized as a fast heart rate arising from the lower chambers of the heart. Programmed ventricular stimulation (PVS) induced non-sustained ventricular tachycardia (VT) in 14% of patients and sustained ventricular arrhythmia in 43% of patients. Sustained ventricular arrhythmia was polymorphic VT in 73% of patients, monomorphic VT in 24% of patients, and ventricular fibrillation in two (3%) patients.

Machine Learned Analysis Modules

In FIG. 1, Analysis system 104 includes the analytical engine comprising the analytics feature analysis module 110 configured to compute features or parameters to generate, via a classifier (e.g., machine-learned classifier), one or more estimated metrics associated with the presence, non-presence, and/or severity of hypertrophic cardiomyopathy. Analysis system 104 may include additional analytical engines configured to perform additional machine-learned-based analyses to assess for other physiological states of a patient, including for the presence, non-presence, and/or severity of other diseases, medical conditions, or an indication thereof, such as (i) heart failure (e.g., left-side or right-side heart failure; heart failure with preserved ejection fraction (HfpEF), heart failure with reduced ejection fraction (HfrEF)), (ii) coronary artery disease (CAD), (iii) various forms of pulmonary hypertension (PH) including, and without limitation, pulmonary arterial hypertension (PAH), (iv) abnormal left ventricular ejection fraction (LVEF), etc. Examples of indicators of certain forms of disease that can be evaluated include the presence or non-presence of elevated or abnormal left-ventricular end-diastolic pressure (LVEDP) as an indication of heart failure or elevated or abnormal mean pulmonary arterial pressure (mPAP) as an indication for pulmonary hypertension.

Table 1 shows a list of classes of features and corresponding descriptions that may be employed for the computation of the estimated metrics associated with the presence, non-presence, and/or severity of hypertrophic cardiomyopathy. The features listed below are also shown in FIG. 2.

TABLE 1

| Feature Class | Description |
|---|---|
| Depolarization and | Quantify the propagative characteristics of the ventricular |
| Repolarization Wave | depolarization (VD) wave and/or ventricular repolarization (VR) |
| Propagation | wave in three-dimensional space, e.g., (1) velocity, (2) |
| | trajectory, (3) orbital frequency (3D rotation rate), and (4) |
| | planarity of the wave. |
| | For example, OVG repolarization abnormality features may |
| | be employed to quantify the repolarization wave amplitude, |
| | duration, relative duration and intervals normalized by predefined |
| | intervals, amplitude to duration ratio, and T-wave area in the time |
| | domain in OVG signal from one or more orthogonal leads (i.e., |
| | orth1, orth2, orth3). T-wave features such as TpTe, the time |
| | duration from the peak to the end of the T-wave, are known to be |
| | elevated in HCM patients as compared to a normal subject [26], |
| | and further, are associated with future risk of ventricular |
| | arrhythmia in HCM patients, and therefore may have some utility |
| | in risk stratification for sudden cardiac death [27]. |
| Depolarization Wave | Quantify the deviations of the VD wave trajectory from the |
| Propagation Deviations | trajectory of the three-dimensional-modeled VD wave to |
| | evaluate the high-frequency & low-amplitude patterns in the VD |
| | trajectory. The model wave is a representation of the VD in a |
| | lower-dimensional space embedding the most prominent |
| | frequency content below 40 Hz. [48] |
| Depolarization Cycle- | Quantify the beat-to-beat variations in cardiac signals. |
| Variability (230) | For example, dynamical (sync) and cycle variability features may |
| | be employed to extract from OVG-SCG, PCG, OVG, PPG, and |
| | respiration signals, and the rate signals may be extracted from |
| | these signals that quantify variabilities across the entire signal or |
| | beat-to-beat variabilities. It is reported that the values of entropy |
| | measures from HRV in the HCM group presented lower values, |
| | indicating a decrease of complexity, than those calculated from |
| | the control group. Moreover, similar behavior was observed |
| | comparing the high and low risk of premature death, the values of |
| | the entropy being lower in high-risk patients [45], [49]. |
| Synchronicity | Quantify variability in registered landmarks in cardiac and PPG |
| | signals and among different SCG signals and/or PCG signals via |
| | a Poincare analysis and/or histogram analysis. |
| | Assess variability in synchronicity between respiratory |
| | inspiration and expiration. |
| | For example, dual-signal (sync) features may be employed |
| | to extract from pairwise OVG-SCG, OVG-PCG, OVG-PPG, and |
| | PPG-SCG. This subset of features may be employed to quantify |
| | the duration and time intervals using the landmarks on OVG, |
| | PPG, SCG, and PCG, such as transit time, systolic time intervals |
| | (STI), and diastolic time intervals (DTI). There have been many |
| | studies of STI demonstrating the close relationship of STI with |
| | other indices of ventricular function [43]. Systolic and diastolic |
| | time intervals can separate patients with low left ventricular |
| | ejection fraction (LVEF) in the HF group. Extracted STIs are the |
| | aortic pre-ejection period (PEP: delay from Q wave of QRS to |
| | aortic valve opening, ms) and LV ejection time (LVET, ms). They |
| | found an AUC of 0.91 for PEP/LVET > 0.43, which allowed for |
| | the detection of LVEF < 35% with a sensitivity of 87% and a |
| | specificity of 84% [44], [50]. |
| Depolarization Dynamical | Quantify the dynamical characteristics of cardiac, PPG, SCG, |
| Systems (DS) (220) | and/or PCG signals, including Lyapunov exponent, correlation |
| | dimension, entropy, mutual information, correlation, and |
| | nonlinear filtering. [51] |
| | Assess change in dynamical characteristics between |
| | respiratory inspiration and expiration. |
| Depolarization and PPG | Quantify properties of the cardiac and SCG signals, such as |
| Linear | waveform amplitudes, durations, heart rate, and morphologies. |
| | Quantify properties of PPG, VPG, and APG signals such as peak |
| | amplitudes, peak-to-peak distances, angles between points, and |
| | various ratios. |
| | For example, OVG, SCG, PCG, and PPG linear features |
| | may be employed to quantify the depolarization and/or |
| | repolarization wave amplitude and their ratios in the OVG signal |
| | from one or more orthogonal leads (i.e., orth1, orth2, orth3), |
| | detected peaks in Red/IR PPG, or in SCG/PCG waveforms. Left |
| | ventricular hypertrophy (LVH) is a common indicator of HCM |
| | that can result in an increased R wave amplitude in the left-sided |
| | ECG leads and an increased S wave depth in the right-sided leads |
| | [24], [52]. |
| | Other examples: OVG linear-LVH features may be |
| | employed to quantify the amplitude of the R and S peaks and their |
| | linear or non-linear intra/inter leads combinations obtained |
| | through transformed 12-lead configuration using six orthogonal |
| | leads described herein. Many examples of EKG voltage criteria |
| | are available in the literature that is commonly used for the |
| | diagnosis of LVH, such as [25]: |
| | Sokolov-Lyon criteria: S wave depth in V1 + tallest R wave |
| | height in V5-V6 > 35 mm. |
| | R wave in lead I + S wave in lead III > 25 mm |
| | R wave in V4, V5, or V6 > 26 mm |
| | Other examples: OVG, SCG, PCG, and PPG linear features |
| | may be employed to quantify the duration of the repolarization |
| | and depolarization waves and the interval between several |
| | fiducials in OVG signal from one or more orthogonal leads (i.e., |
| | orth1, orth2, orth3) or wave duration and intervals in Red/IR PPG, |
| | or in SCG/PCG waveforms. The thickened LV wall can lead to |
| | prolonged depolarization (increased R wave peak time) and |
| | delayed repolarization (ST and T-wave abnormalities) in the |
| | lateral leads. Ventricular preexcitation manifests on ECG with a |
| | short PR interval may be associated with structural heart diseases |
| | HCM [28], [52]. |
| | Other examples: PPG linear features may be employed to |
| | quantify systolic ejection time, angle of systolic rise, angle of |
| | diastolic decline, and respiratory variation. Green et al. [33] may |
| | use duration, slope, and respiration features extracted from PPG |
| | signals to separate HCM from healthy controls. Kleid et al. [34] |
| | examined nocturnal oximetry signals in 100 HCM subjects. 71% |
| | of subjects had signal abnormalities suggestive of obstructive |
| | sleep apnea. |
| | Assess change in SCG/PCG signals between respiratory |
| | inspiration and expiration. |
| Wavelet (222) | Quantify the main frequency components of cardiac, PPG, SCG, |
| | and/or PCG signals using wavelet analysis. |
| | OVG, SCG, or PCG Wavelet features may be employed to |
| | quantify characteristics of the energy distribution in time and |
| | frequency within a high-energy content region in the power |
| | spectrum associated with atrial and ventricular depolarization and |
| | repolarization waves in OVG signal in one or more orthogonal |
| | leads, or PPG and SCG isolated waveforms. With possible |
| | hypertrophic ventricles and ventricular arrhythmias in HCM, |
| | intensified and skewed energy distribution (for asymmetrical VH) |
| | is expected to be observed in the time and frequency domain in |
| | wavelet spectrum representation of one or more orthogonal leads |
| | (i.e., orth1, orth2, orth3). Utility of the continuous wavelet |
| | spectrum of cardiac signals in the detection of VH and wavelet |
| | decomposition in the detection of ventricular fibrillation with |
| | non-sustained ventricular tachycardia in patients with |
| | hypertrophic cardiomyopathy is reported in [29], [30], [53]. |
| | Assess change in main frequency components SCG and/or |
| | PCG signals between respiratory inspiration and expiration. |
| Power Spectral Density | Quantify the power spectrum and frequency content of the |
| (222) | biophysical signals (e.g., PPG, cardiac, SCG, and/or PCG |
| | signals) using power spectrum and coherence (cross-spectral |
| | analysis) analysis. |
| | OVG PSD features may be employed to quantify the power |
| | of an OVG, PPG, SCG, or PCG signal in one or more orthogonal |
| | leads or channels and their relative ratios. With the intensified |
| | and prolonged depolarization wave due to the hypertrophic |
| | ventricles in HCM, more energy is expected to be captured in the |
| | filtered power spectrum in the OVG signal from one or more |
| | orthogonal leads (i.e., orth1, orth2, orth3) and/or the SCG/PCG |
| | signals. [47] |
| | Assess change in the spectrum and frequency content of the |
| | SCG signals between respiratory inspiration and expiration. |
| Visual (224) | Quantify properties of PPG, cardiac, SCG, and/or PCG signals |
| | over loop regions (e.g., atrial depolarization, ventricular |
| | depolarization, and ventricular repolarization) in 3D phase |
| | spaces, projections thereof, and loop vectors. PPG is analyzed in |
| | 3D space using PPG, VPG, and APG. |
| | For example, OVG Visual features may be employed to |
| | quantify the shape of the QRS and T loops in phase space |
| | representation of the signals, such as maximal vector, perimeter, |
| | the area in three-dimension, and different quadrants as well as |
| | their two-dimensional projection on cartesian planes. |
| | Other examples: OVG Visual features quantifying the |
| | morphology of QRS loop in phase space representation of the |
| | signals, such as axis deviations associated with maximal QRS |
| | vector in three-dimension, its two-dimensional projection on |
| | cartesian planes, as well as geometrical factors including |
| | eccentricity, curvature. Several hallmarks on vectorcardiogram |
| | (VCG) were reported to be indicative of LVH [35]. It was |
| | reported that the T wave vector magnitude value was |
| | significantly smaller in HCM patients compared to healthy |
| | controls [36]. Possible deviations in the orientation of the QRS |
| | loop on VCG and the QRS initial vector (within the first 20-30 |
| | ms) in three-dimensional space are reported in [37], [54]. |
| | Assess change in loop regions of 3D data derived from SCG |
| | and/or PCG, 1^{st} der. SCG and/or PCG, and 2^{nd} derv. SCG |
| | and/or PCG between respiratory inspiration and expiration. |
| Respiration (226) | Approximate a respiration waveform using either (i) PPG and |
| | cardiac signals or (ii) SCG signals to assess (1) heart rate |
| | variability, (2) respiration rate, (3) discrepancy features |
| | representing the distance between respiration and modulation |
| | signals, (4) square coherence representing the correlation |
| | between modulation and respiration rate signals. |
| | For example, respiration features may be extracted from |
| | the respiration signals derived from amplitude or frequency |
| | modulations in OVG, PPG signals. This subset of features may |
| | be employed to quantify the respiration rate, respiration |
| | variabilities in the time and frequency domain, and cross- |
| | spectral agreement between proxy respiration derived from each |
| | signal in different channels. Automated analysis showed a |
| | significant difference in oHCM patients for morphometric PPG |
| | pulse wave features, including measures of the systolic ejection |
| | time, rate of rising during systole, and respiratory variations |
| | [38]. In addition, two murmurs are often cited as being present in |
| | patients with HCM. The first murmur is because of systolic |
| | anterior motion of the mitral valve leading to poor leaflet |
| | coaptation and mitral regurgitation. The second murmur is |
| | because of turbulent flow through the outflow tract and is |
| | present as a mid-systolic, which can mimic the murmur of aortic |
| | stenosis [39]. It is reported that a piezoelectric sensor can be |
| | used to capture signals, similar to SCG and/or PCG, and detect |
| | heart murmurs objectively [40]. It is contemplated that the |
| | difference in the blood flow rate during the inspiration and |
| | expiration, manifesting itself in SCG-respiratory signal, could |
| | capture the murmur effects in HCM patients. |
| | Other examples: heart rate variability (HRV) features may |
| | be employed to extract from the heart rate signals derived from |
| | OVG, PPG, SCG, and PCG signals. This subset of features may |
| | be employed to quantify the heart rate and heart rate variabilities |
| | in time (using statistical analysis) and frequency domain. Folino |
| | et al. showed a significant correlation between the time domain |
| | HRV features and its correlations with ventricular arrhythmias, |
| | heart function, and prognostic outcome in patients with |
| | arrhythmogenic right ventricular cardiomyopathy [41]. Another |
| | study reported that Patients with idiopathic dilated |
| | cardiomyopathy, even those without congestive heart failure, |
| | had significantly lower values for HRV than those of control |
| | subjects, which is mainly related to left ventricular dysfunction |
| | and not to ventricular arrhythmias [42], [55]. |
| | For HCM assessment, these features can include generating |
| | delineated inspiration and expiration portions of the SCG |
| | and/or PCG signals employed for the analysis. |
| Physiological | Quantify physiological aspects of the cardiac, SCG, and/or PCG |
| | signals. |
| | For example, OVG Physiological features may be employed |
| | to implement criteria for determining left, right, and bi-atrial |
| | enlargement. Criteria use lead-specific combinations of duration, |
| | morphology, and amplitude as inputs. Causes of LAE may include |
| | HCM [31], whereas RAE is mainly caused by pulmonary |
| | hypertension [32]. The causes of biatrial enlargement contain both |
| | LAE and RAE causes. [56] |
| LVET Estimation (228) | Approximate LVET using SCG and/or PCG signals. |

Detailed descriptions of some of the analyses of Table 1, as could be applied to SCG signals and for the determination of the presence, non-presence, and/or severity of HCM, may be found in [1]-[13] and [20]-[56], among others, each of which is hereby incorporated by reference herein in its entirety.

Other feature modules which may be employed are described in U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. patent application Ser. Nos. 16/831,264; 16/831,380; 17/132,869; PCT Application Nos. PCT/IB2020/052889; PCT/IB2020/052890, each of which is hereby incorporated by reference herein in its entirety.

The analysis and associated modules (e.g., 110) may evaluate the provided features in relation to SCG or phonocardiographic signals, in addition to the PPG and cardiac signals, as well as extend the various analyses (frequency, dynamics, cycle variability, etc., as noted above), to assess for changes in the values of the features between inspiration and expiration portion of the signals.

The modules (e.g., 110) may include an analysis for the estimation of LVET or a parameter correlated to elevated LVET.

MEMS Accelerometer System

In the example shown in FIG. 1, measurement system 102 includes a chest-worn or mounted SCG/PCG measurement device 118 configured with two or more MEMS accelerometers, transducers, or sensors to provide at least two or more SCG/PCG measurements at least at an apex region of the heart and base region. The SCG/PCG device 118 may be connected to a biophysical signal capture system 120 configured to measure cardiac signals via a set of surface electrodes 122 (shown as 122a-122f) and/or photoplethysmographic signals via a PPG sensor device 124. Example wireless communication operation and circuitry are described in [14], which is hereby incorporated by reference herein in its entirety.

The SCG/PCG measurement device 118 includes a first MEMS accelerometer, transducer, or sensor 126 and a second MEMS accelerometer, transducer, or sensor 128. The two or more MEMS accelerometers, transducers, or sensors can provide seismocardiogram signals to the analysis system, to which phase differences and differential evaluation of the signals can be performed by the analytical engine. For example, the first MEMS accelerometer, transducer, or sensor 126 can be configured to be positioned proximal to the apex 130, and the second MEMS accelerometer, transducer, or sensor 128 is configured to be positioned near or at a base region 132 of the left ventricle. In the example shown in FIG. 1, device 118 and associated sensors 126 and 128 are non-invasively placed on the skin of a subject at illustratively shown regions. The cross-section of the heart is shown in FIG. 1 merely to provide illustrative placement of the sensors in relation to certain heart structures in certain embodiments.

Example Biophysical Signals for Hypertrophic Cardiomyopathy Assessment

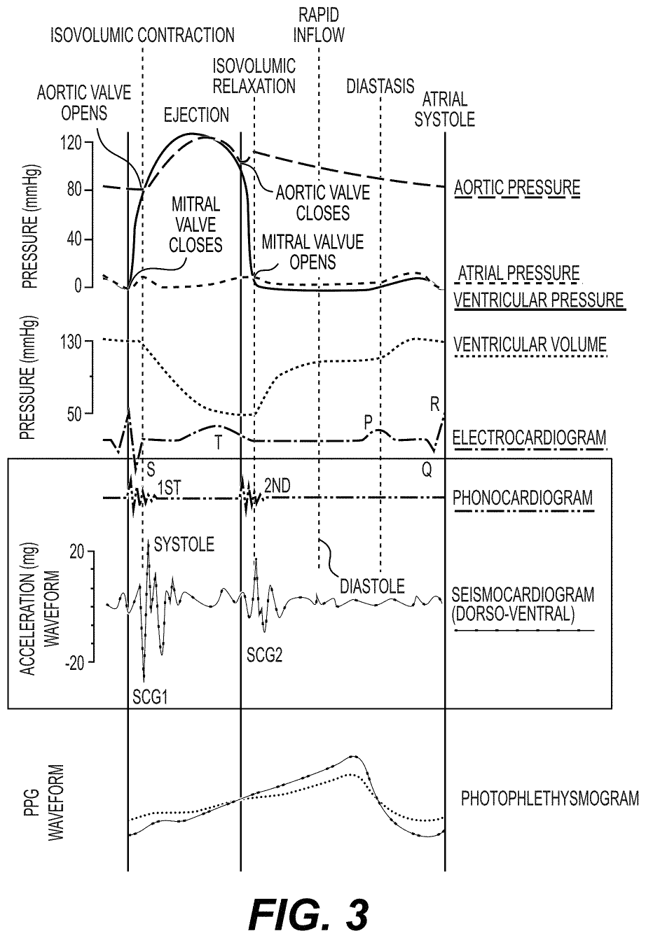

FIG. 3 shows a diagram of example biophysical signals acquired for the evaluation of hypertrophic cardiomyopathy in accordance with an illustrative embodiment. In the example shown in FIG. 3, an example seismocardiographic signal, phonocardiographic signal, cardiac signal (shown as “Electrocardiogram”), and photoplethysmographic signal are shown as being concurrently acquired over one heart cycle. The diagram further illustrates an example of aortic pressure, atrial pressure, ventricular volume, and ventricular pressure over the same heart cycle. Registration/landmark points may be determined in a single signal (e.g., peaks of a cardiac waveform, seismocardiographic, and/or phonocardioagraphic signals) or among signals (e.g., cross-over points between two photoplethysmographic waveforms, two seismocardiographic waveforms, etc.) to which synchronicity and phase can be assessed in relation to other acquired biophysical signals; e.g., as described in U.S. Publication No. 2020/0397324 entitled “Method and System to Assess Disease Using Dynamical Analysis of Cardiac and Photoplethysmographic Signals,” which is hereby incorporated by reference herein in its entirety.

Example SCG/PCG Measurement System #1

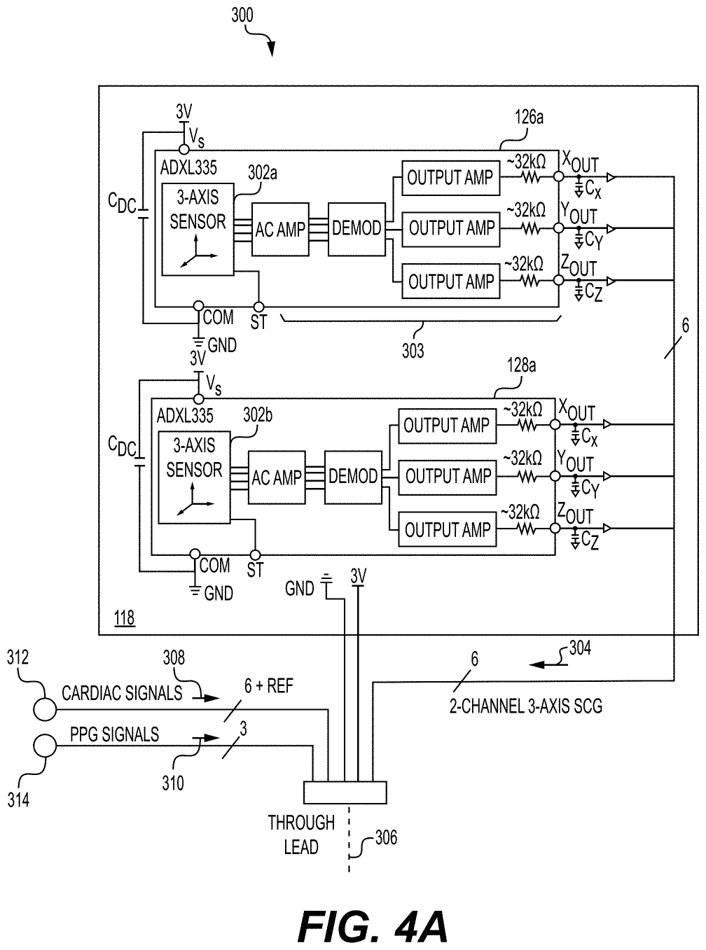

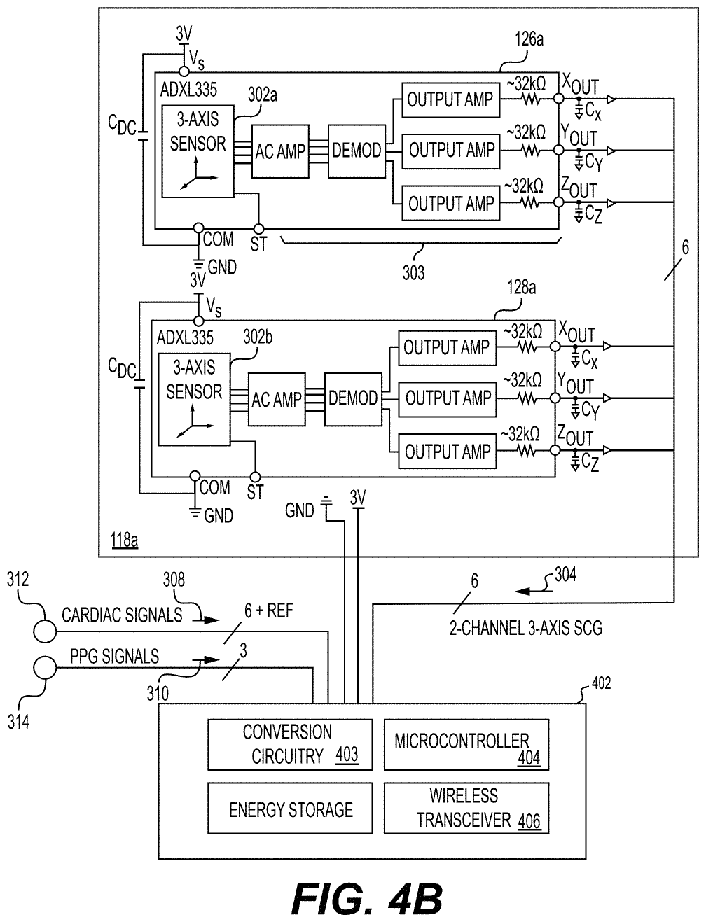

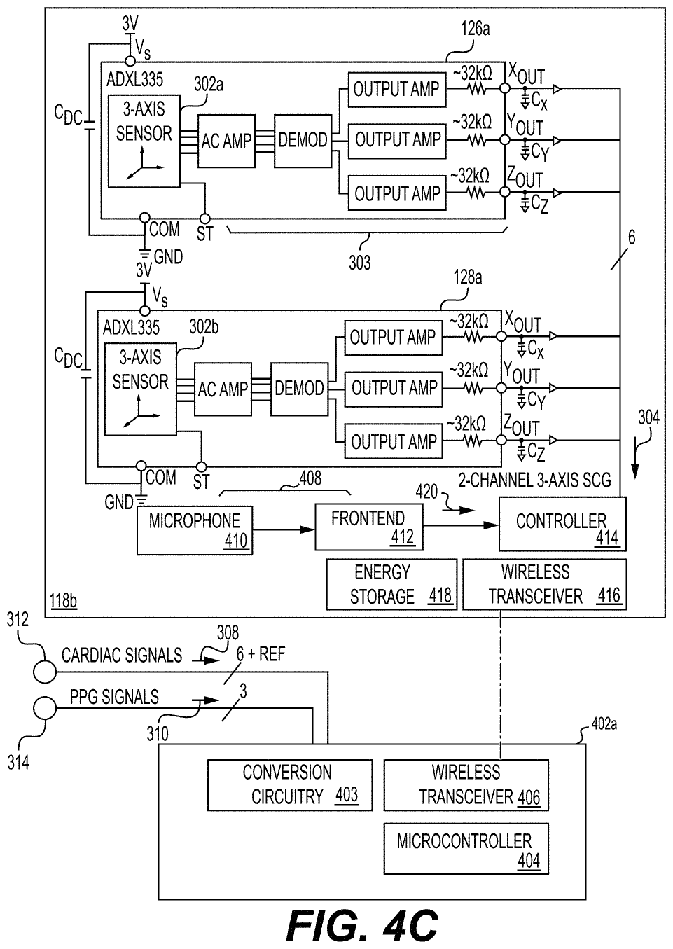

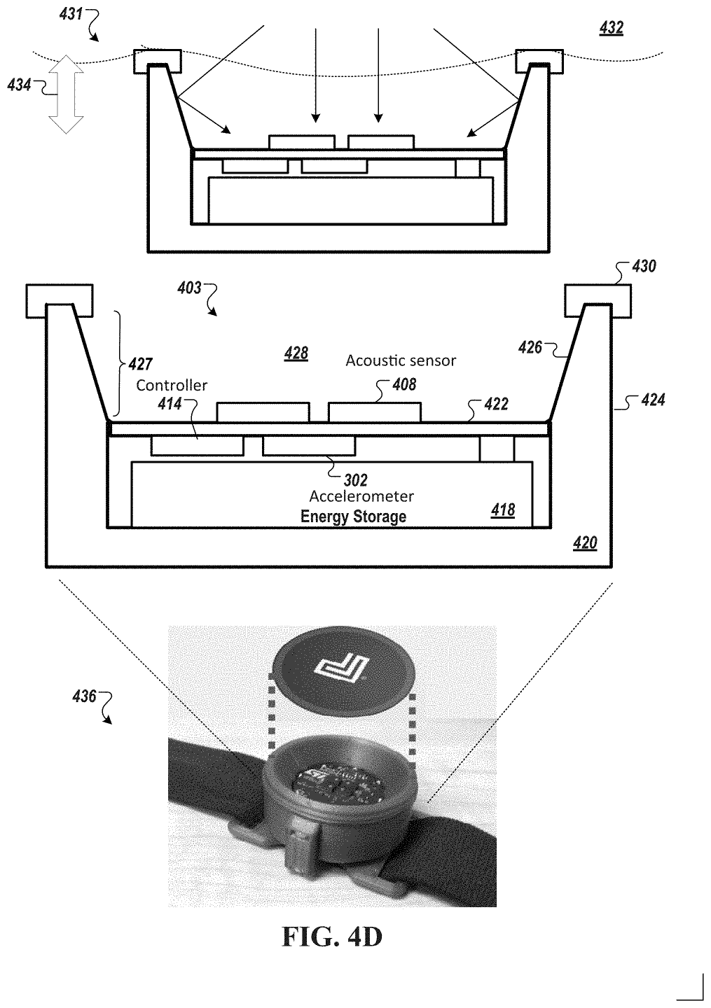

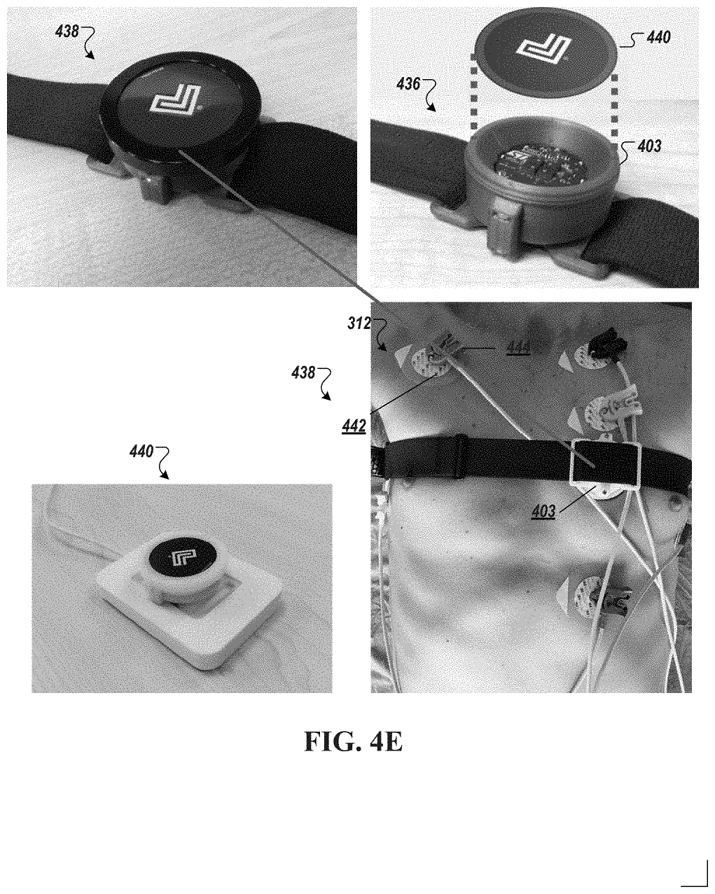

FIG. 4A shows a diagram of an example SCG/PCG measurement device 118 in accordance with an illustrative embodiment. In the example shown in FIG. 4A, the SCG/PCG measurement device 118 includes two or more MEMS accelerometers, transducers, or sensors 126, 128 (shown as 126a, 128a). The MEMS accelerometer, transducer, or sensors 126a, 128a, in this example, include a 3-axis accelerometer 302 (shown as 302a, 302b), e.g., the small, low-power ADXL335 3-axis accelerometer manufactured by Analog Devices of Wilmington Mass. that is integrated into a small 4 mm×4 mm×1.45 mm package IC. The 3-axis accelerometers 302a, 302b are coupled amplifiers and filters 303 to provide seismographic signals 304 to a cable 306 that connects to the measurement system 102. The cardiac signals 308, in the example, are acquired at each of six surface electrodes 312 and are carried over six conductors through cable 306 to the measurement system 102. A reference electrode is also employed. The photoplethysmographic signals 310, in the example, is generated at PPG lead snap 314, as the photoplethysmogram sensor is carried over three conductors (two signals and GND) through cable 306 to the measurement system 102. The measurement system 102 includes conversion circuitries to convert and digitize the acquired biophysical signals. In some embodiments, the surface electrodes 312 and/or PPG lead snap 314 may include accelerometers, e.g., as described in [14] (A4L BCG application), which is hereby incorporated by reference herein in its entirety.

MEMS accelerometers and associated acquisition circuitries are configured to have a measurement range of at least ±1 g or ±2 g and a bandwidth of up to 1 kHz. In some embodiments, the SCG/PCG measurement device 118 is configured with MEMS accelerometers having a sensitivity of at least 0.164 μs/μg and a noise density below 6.5 μg/Hz. While in the example of FIG. 4A, the MEMS accelerometers and associated acquisition circuitries are configured for unipolar outputs, in other embodiments, the MEMS accelerometers and associated acquisition circuitries are configured for bipolar and/or differential operations. Additional configurations of the MEMS accelerometers and associated acquisition circuitries that may be employed are described in [15] (sensor manuscript), which is hereby incorporated by reference herein in its entirety.

In some embodiments, the MEMS sensor includes an acoustic sensor or transducer, e.g., acoustic respiration sensors (e.g., model. RAS-45, manufactured by Masimo, Corp., Irvine, Calif.). Another example of the acoustic-based sensor is the sensor employed in a digital stethoscope system such as the ECG+Digital Stethoscope (e.g., the DUO ECG+Digital Stethoscope manufactured by Eko Devices, Inc., Oakland, Calif.).

In some embodiments, the MEMS sensor includes an accelerometer-based sensor, e.g., a 9DoF inertial sensing comprising an accelerometer, gyroscope, magnetic, and pressure sensor (e.g., Shimmer3 IMU, manufactured by Shimmer, Cambridge, Mass.). Another example of an accelerometer-based sensor is an IMU sensor (Movesense IMU manufactured by Movesense, Vantaa Finland) comprising a 9-axis motion sensor that includes acceleration, gyro, and magnetometer. An example of an accelerometer-based sensor is an IMU tracking sensor (ICM-20948 manufactured by TDK, InvenSense, San Jose, Calif.) comprising a 9-axis motion sensor that includes acceleration, gyro, and magnetometer. An example of an accelerometer-based sensor is an IMU tracking sensor (Sense Connect Detect model no. SCD110 manufactured by Bosch Connected Devices and Solutions GmbH (Germany)).

These MEMS sensors (acoustics, accelerometers, IMUs) can be used alone or in any combination with one or more other sensors, including but not limited to our two current sensor types or other sensor types described herein, to gather information for generating diagnostic tools for any indication for at least one of HCM, HF, PH, CAD, or a combination thereof, in their various forms, or associated conditions or indications. In addition, these MEMS sensors may be used for generating diagnostic tools for any indication described herein that are not HCM, HF, PH, and CAD in their various forms.

FIG. 4B shows the example SCG/PCG measurement device 118 (shown as 118a) of FIG. 4A configured as a wireless measurement module comprising a wireless transceiver module 402. The wireless transceiver module 402 includes front-end conversion circuitries 403, a microcontroller 404, and a wireless transceiver circuit 406. The wireless transceiver circuit 406 can interface over a wireless connection to an interface device.

The cardiac signals 308 and photoplethysmographic signals 310 may be acquired using circuitries and computing hardware, software, firmware, middleware, etc., in a biophysical signal capture system described in U.S. Pat. No. 10,542,898, entitled “Method and Apparatus for Wide-Band Phase Gradient Signal Acquisition,” or U.S. Patent Publication No. 2018/0249960, entitled “Method and Apparatus for Wide-Band Phase Gradient Signal Acquisition,” each of which is hereby incorporated by reference herein in its entirety.

Other configurations and topologies, as discussed herein, may be employed, e.g., MEMS microphones or acoustic transducers, among others.

Example SCG/PCG Measurement System #2

FIG. 4C shows a diagram of another example SCG/PCG measurement device 118 (shown as 118b) in accordance with an illustrative embodiment. In the example shown in FIG. 4C, the SCG/PCG measurement device 118b includes a phonocardiogram device 408 (comprising an acoustic transducer) and MEMS accelerometers in a wireless module (shown as 403 in FIG. 4D).

The phonocardiogram device 408 comprises one or more microphones 410 and a microphone frontend circuitries 412. The microphone frontend circuitries 412 include conversion, filters, and amplifier circuitries to convert and digitize the acquired biophysical signals. The microphone 410 and microphone frontend 412 are configured to capture acoustic signatures of the heart and nearby structures and murmurs. In some embodiments, the phonocardiogram device 408 is configured to acquire the acoustic signal has a rate of at least 8 kHz with a resolution of 12 bits. Other sampling rates and resolutions may be employed. Other examples of phonocardiogram device 408 include acoustic respiration sensors (e.g., model. RAS-45, manufactured by Masimo, Corp., Irvine, Calif.) or digital stethoscope systems such as the ECG+Digital Stethoscope (e.g., the DUO ECG+Digital Stethoscope manufactured by Eko Devices, Inc., Oakland, Calif.).

In the example shown in FIG. 4C, the SCG/PCG measurement device 118b includes a controller 414, wireless transceiver 416, and energy storage 418. The SCG/PCG measurement device 118b is configured as a wireless measurement module, e.g., as a wearable device as described herein, comprising the wireless transceiver module 416 that communicates with a base station 402 (shown as 402a) comprising the wireless transceiver 406. In this configuration, the base station 402a includes front-end conversion circuitries 403 (for the acquisition of the cardiac signals 308 and PPG signal 310), the microcontroller 404, and the wireless transceiver circuit 406.

As described in relation to FIG. 4B, the MEMS accelerometers and associated acquisition circuitries may be configured to have a measurement range of at least ±1 g or ±2 g and a bandwidth of up to 1 kHz. In some embodiments, the SCG/PCG measurement device (e.g., 118b) is configured with MEMS accelerometers having a sensitivity of at least 0.164 μs/μg and a noise density below 6.5 μg/Hz. The MEMS sensor may include a 9-DoF inertial sensing comprising an accelerometer, a gyroscope, a magnetic sensor, and a pressure sensor (e.g., Shimmer3 IMU, manufactured by Shimmer, Cambridge, Mass.) or an IMU sensor (Movesense IMU manufactured by Movesense, Vantaa Finland) comprising a 9-axis motion sensor that includes an accelerometer, a gyroscope, and a magnetometer. An example of an accelerometer-based sensor is an IMU tracking sensor (ICM-20948 manufactured by TDK, InvenSense, San Jose, Calif.) comprising a 9-axis motion sensor that includes an accelerometer, a gyroscope, and a magnetometer. An example of an accelerometer-based sensor is an IMU tracking sensor (Sense Connect Detect model no. SCD110 manufactured by Bosch Connected Devices and Solutions GmbH (Germany)).

These MEMS sensors (acoustics, accelerometers, IMUs) can be used alone or in any combination with one or more other sensors, including but not limited to sensor types described herein, to gather information for generating diagnostic tools for any indication for at least one of HCM, HF, PH, CAD, or any combination thereof, in their various forms, or associated conditions or indications. In addition, these MEMS sensors may be used for generating diagnostic tools for any indication described herein that are not HCM, HF, PH, and CAD in their various forms.

The cardiac signals 308 and photoplethysmographic signals 310 may be acquired using circuitries and computing hardware, software, firmware, middleware, etc., in a biophysical signal capture system described in U.S. Pat. No. 10,542,898, entitled “Method and Apparatus for Wide-Band Phase Gradient Signal Acquisition,” or U.S. Patent Publication No. 2018/0249960, entitled “Method and Apparatus for Wide-Band Phase Gradient Signal Acquisition,” each of which is hereby incorporated by reference herein in its entirety.

During signal acquisition, a patient may be patted (e.g., firmly tapped) to provide a spike in the measurement that can be used to synchronize the measurements between the two acquisition systems 402 and 118b.

Example Wearable MEMs Sensor

FIG. 4D is a diagram of an example measurement system 102 (shown as 403) configured as a wearable MEMS sensor device 403. The wearable MEMS sensor device 403 includes a housing 420 configured to house one or more electronic boards 422 comprising the MEMS accelerometers 302 (shown as “Accelerometer” 302), the phonocardiogram device 408 (shown as “Acoustic Sensor” 408), controller 414, wireless transceiver 416 (integrated with 414), and energy storage 418, e.g., as described in relation to FIG. 4C.

In the example shown in FIG. 4D, the housing 420 has an outer wall 424 and an inner wall 426. The inner wall 426 has a tapered region 427 that defines and focuses sound (see diagram 431) at the entry region 428 of the housing 420 to the phonocardiogram device 408. The phonocardiogram device 408 is located on the portion of the electronic board 422 to face the entry region 428. The entry region 428 can form a volume defined by the housing surfaces and the body to direct and maintain acoustic energy in said volume. The housing 420 further includes an elastomeric member 430 that can serve as a gasket-type interface to adhere and maintain contact (434) with the body (shown as 432). Diagram 436 (see also FIG. 4E) is an image of an example fabricated wearable MEMS sensor device 403.

FIG. 4E shows images of the example fabricated wearable MEMs sensor device 403 of FIG. 4D, configured as a wearable chest sensor device. In FIG. 4E, diagram 438 shows another example of the example fabricated wearable MEMS sensor device 403 with a front plate 440 placed over the entry region 428. Diagram 438 shows an example placement of the wearable MEMS sensor device 403 on a person. As shown in diagram 438, the wearable MEMS sensor device 403 is placed with the entry region 428 directed toward the person and can be placed over the heart (as shown) and in various locations of the body as described herein. Diagram 438 also shows the surface electrodes 312. The electrodes 312 are each an ECG pad 442 that is coupled to a clip connector 444.

Diagram 440 shows the wearable MEMS sensor device 403 placed on a wireless rechargeable station. Examples of placement are described in U.S. Pat. No. 10,542,898, which is hereby incorporated by reference herein in its entirety.

Example HCM Treatment

Following the generation of the estimation of the metrics associated with the presence or non-presence of hypertrophic cardiomyopathy, the generated estimation can be used provided to a patient's report, e.g., in a healthcare portal, or as output to a wearable device or as output to a piece of medical equipment for the treatment of a disease, condition, or indication of either.

Treatment for HCM can include medication or surgery. Pharmacological treatment can include the administration of beta-blockers (e.g., metoprolol, propranolol, or atenolol), calcium channel blockers (e.g., verapamil or diltiazem), heart rhythm drugs (e.g., amiodarone or disopyramide), mavacamten, among others. Surgical intervention can include septal myectomy to remove a part of the thickened, overgrown septum wall, apical myectomy to remove thickened heart muscle from near the tip of the heart, septal ablation to destroy a part of the thickened heart muscle, or implantable cardioverter-defibrillator (ICD) to continuously monitor the heartbeat.

While the present disclosure is directed to the practical assessment of biophysical signals, e.g., raw or pre-processed photoplethysmographic signals, biopotential/cardiac signals, seismocardiographic signals, phonocardiographic signals, etc., in the diagnosis and treatment of cardiac-related pathologies and conditions, such assessment can be applied to the diagnosis, treatment, and tracking/monitoring (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications.

The exemplary biophysical sensor system may be implemented as a modular medical evaluation system [18], which is hereby incorporated by reference herein in its entirety.

Example Clinical Evaluation System

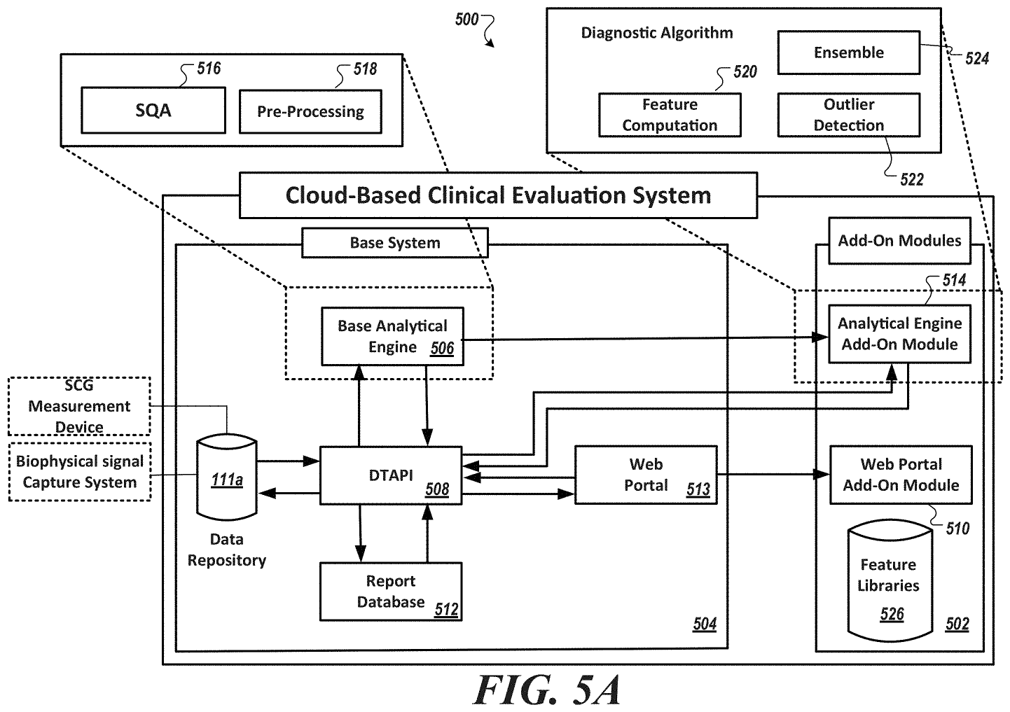

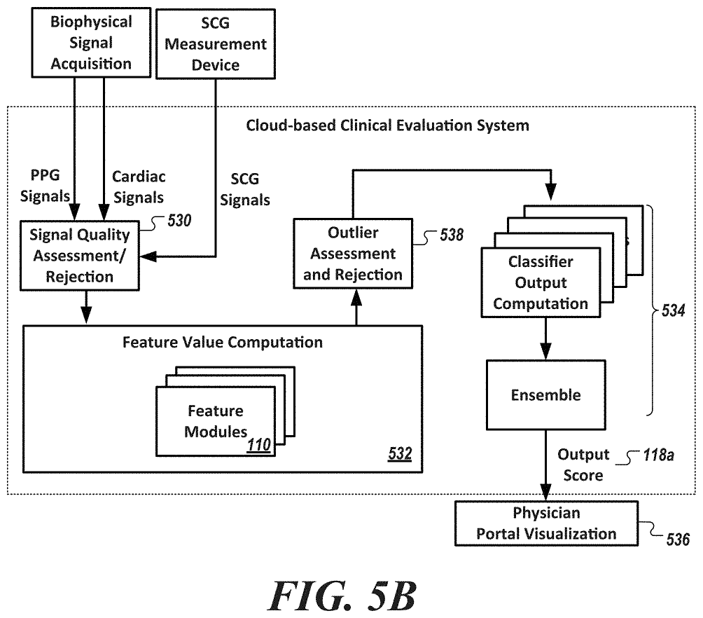

FIG. 5A shows an example clinical evaluation system 500 (also referred to as a clinical and diagnostic system) that implements the modules of FIG. 1 to non-invasively perform machine-learned-based analyses to generate, via a classifier (e.g., machine-learned classifier), one or more metrics associated with the HCM-associated state of a patient or subject according to an embodiment. Indeed, the feature modules (e.g., of FIGS. 1, 5-14) can be generally viewed as a part of a system (e.g., the clinical evaluation system 500) in which any number and/or types of features may be utilized for a disease state, medical condition, an indication of either, or combination thereof that is of interest, e.g., with different embodiments having different configurations of feature modules. This is additionally illustrated in FIG. 5A, where the clinical evaluation system 500 is of a modular design in which disease-specific add-on modules 502 (e.g., to assess for HCM, elevated LVEDP or mPAP, CAD, PH/PAH, abnormal LVEF, hFpEF, and others described herein) are capable of being integrated alone or in multiple instances with a singular platform (i.e., a base system 504) to realize system 500's full operation. The modularity allows the clinical evaluation system 500 to be designed to leverage the same synchronously acquired biophysical signals and data set and base platform to assess for the presence of several different diseases such as disease-specific algorithms are developed, thereby reducing testing and certification time and cost.

In various embodiments, different versions of the clinical evaluation system 500 may implement the assessment system 103 (FIG. 1) by having included containing different feature computation modules that can be configured for a given disease state(s), medical condition(s), or indicating condition(s) of interest. In another embodiment, the clinical evaluation system 500 may include more than one assessment system 103 and may be selectively utilized to generate different scores specific to a classifier 112 of that engine 103. In this way, the modules of FIGS. 1 and 5, in a more general sense, may be viewed as one configuration of a modular system in which different and/or multiple engines 103, with different and/or multiple corresponding classifiers 112, may be used depending on the configuration of the module desired. As such, any number of embodiments of the modules of FIG. 1 may exist.

In FIG. 5A, System 500 can analyze one or more biophysical-signal data sets (e.g., 110) using machine-learned disease-specific algorithms to assess for the likelihood of elevated LVEDP, as one example, of pathology or abnormal state. System 500 includes hardware and software components that are designed to work together in combination to facilitate the analysis and presentation of an estimation score using the algorithm to allow a physician to use that score, e.g., to assess for the presence, non-presence, and/or severity of a disease state, medical condition, or an indication of either.

The base system 504 can provide a foundation of functions and instructions upon which each add-on module 502 (which includes the disease-specific algorithm) then interfaces to assess for the pathology or indicating condition. The base system 504, as shown in the example of FIG. 5A, includes a base analytical engine or analyzer 506, a web-service data transfer API 508 (shown as “DTAPI” 508), a report database 510, a web portal service module 513, and a data repository 111 (shown as 111a).

Data repository 111a, which can be cloud-based, stores data from the signal capture system 102 (shown as 102b). Biophysical signal capture system 102b, in some embodiments, is a reusable device designed as a single unit with a seven-channel lead set and photoplethysmogram (PPG) sensor securely attached (i.e., not removable). Signal capture system 102b, together with its hardware, firmware, and software, provides a user interface to collect patient-specific metadata entered therein (e.g., name, gender, date of birth, medical record number, height, and weight, etc.) to synchronously acquire the patient's electrical and hemodynamic signals. The signal capture system 102b may securely transmit the metadata and signal data as a single data package directly to the cloud-based data repository. The data repository 111a, in some embodiments, is a secure cloud-based database configured to accept and store the patient-specific data package and allow for its retrieval by the analytical engines or analyzer 506 or 514.

Base analytical engine or analyzer 506 is a secure cloud-based processing tool that may perform quality assessments of the acquired signals (performed via “SQA” module 516), the results of which can be communicated to the user at the point of care. The base analytical engine or analyzer 506 may also perform pre-processing (shown via pre-processing module 518) of the acquired biophysical signals (e.g., 110—see FIG. 1). Web portal 513 is a secure web-based portal designed to provide healthcare providers access to their patient's reports. An example output of the web portal 513 is shown by visualization 536. The report databases (RD) 512 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient's general health record. In some embodiments, web portal 513 is accessed by a call center to provide the output clinical information over the telephone. Database 512 may be accessed by other systems that can generate a report to be delivered via the mail, courier service, facsimile, personal delivery, etc.

Add-on module 502 includes a second part 514 (also referred to herein as the analytical engine (AE) or analyzer 514 and shown as “AE add-on module” 514) that operates with the base analytical engine (AE) or analyzer 506. Analytical engine (AE) or analyzer 514 can include the main function loop of a given disease-specific algorithm, e.g., the feature computation module 520, the classifier model 524 (shown as “Ensemble” module 524), and the outlier assessment and rejection module 524 (shown as “Outlier Detection” module 524). In certain modular configurations, the analytical engines or analyzers (e.g., 506 and 514) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient's score using the disease-specific algorithm. To execute the analysis pipeline, AE add-on module 514 can include and execute instructions for the various feature modules 110 and classifier module 112 as described in relation to FIG. 1 to determine an output score (e.g., 116) of the metrics associated with the physiological state of a patient. The analysis pipeline in the AE add-on module 514 can compute the features or parameters (shown as “Feature Computation” 520) and identifies whether the computed features are outliers (shown as “Outlier Detection” 522) by providing an outlier detection return for a signal-level response of outlier vs. non-outlier based on the feature. The outliers may be assessed with respect to the training data set used to establish the classifier (of module 112). AE add-on module 514 may generate the patient's output score (e.g., 116) (e.g., via classifier module 524) using the computed values of the features and classifier models. In the example of an evaluation algorithm for the estimation of HCM, the output score (e.g., 116) is an HCM score.

The clinical evaluation system 500 can manage the data within and across components using the web-service DTAPIs 508 (also may be referred to as HCPP web services in some embodiments). DTAPIs 508 may be used to retrieve acquired biophysical data sets from and to store signal quality analysis results to the data repository 111a. DTAPIs 508 may also be invoked to retrieve and provide the stored biophysical data files to the analytical engines or analyzers (e.g., 506, 514), and the results of the analytical engine's analysis of the patient signals may be transferred using DTAPI 508 to the report database 510. DTAPIs 508 may also be used, upon a request by a healthcare professional, to retrieve a given patient data set to the web portal module 513, which may present a report to the healthcare practitioner for review and interpretation in a secure web-accessible interface.

Clinical evaluation system 500 includes one or more feature libraries 526 that store the machine-learned-based analyses, e.g., as features. The feature libraries 526 may be a part of the add-on modules 502 (as shown in FIG. 5A) or the base system 504 (not shown) and are accessed, in some embodiments, by the AE add-on module 514.

Example Operation of the Modular Clinical Evaluation System

FIG. 5B shows a schematic diagram of the operation and workflow of the analytical engines or analyzers (e.g., 506 and 514) of the clinical evaluation system 500 of FIG. 5A in accordance with an illustrative embodiment.

Signal quality assessment/rejection (530). Referring to FIG. 5B, the base analytical engine or analyzer 506 assesses (530), via SQA module 516, the quality of the acquired biophysical-signal data set while the analysis pipeline is executing. The results of the assessment (e.g., pass/fail) are immediately returned to the signal capture system's user interface for reading by the user. Acquired signal data that meet the signal quality requirements are deemed acceptable (i.e., “pass”) and further processed and subjected to analysis for the presence of metrics associated with the pathology or indicating condition (e.g., HCM, elevated LVEDP or mPAP, CAD, PH/PAH, abnormal LVEF, and/or hFpEF) by the AE add-on module 514. Acquired signals deemed unacceptable are rejected (e.g., “fail”), and a notification is immediately sent to the user to inform the user to immediately obtain additional signals from the patient (see FIG. 2).

The base analytical engine or analyzer 506 performs two sets of assessments for signal quality, one for the electrical signals and one for the hemodynamic signals. The electrical signal assessment (530) confirms that the electrical signals are of sufficient length, that there is a lack of high-frequency noise (e.g., above 170 Hz), and that there is no power line noise from the environment. The hemodynamic signal assessment (530) confirms that the percentage of outliers in the hemodynamic data set is below a pre-defined threshold and that the percentage and maximum duration that the signals of the hemodynamic data set are railed or saturated is below a pre-defined threshold.

Feature Value Computation (532). The AE add-on module 514 performs feature extraction and computation to calculate feature output values. In the example of the LVEDP algorithm, the AE add-on module 514 determines, in some embodiments, feature outputs belonging to different feature families (e.g., generated in modules 110).

Additional descriptions of the various feature to which the HCM algorithm and associated machine-learning analyzes may be based is provided [1], [2], [3], [4], [5], [6], [7], [8], [12], [13], [19], each of which is hereby incorporated by reference herein in its entirety.

Classifier Output Computation (534). The AE add-on module 514 then uses the calculated feature outputs in classifier models (e.g., machine-learned classifier models) to generate a set of model scores. The AE add-on module 514 may join the set of model scores, e.g., in an ensemble of the constituent models, which, in some embodiments, averages the output of the classifier models.

In some embodiments, classifier models may include models that are developed based on ML techniques described in U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety. Another example of a training system that may be used to configure the analytical engine is described in [19], which is hereby incorporated by reference herein in its entirety.

In the example, machine-learned classifier models may employ ElasticNet machine-learned classifier models, RandomForest machine-learned classifier models, and extreme gradient boosting (XGB) classifier models, among others, including those described herein. In some embodiments, the patient's metadata information, such as age, gender, and BMI value, may be used. The output of the ensemble estimation may be a continuous score.

Physician Portal Visualization (536). The patient's report may include a visualization 536 of the acquired patient data and signals and the results of the disease analyses. The analyses are presented, in some embodiments, in multiple views in the report. A healthcare provider, e.g., a physician, can review the report and interpret it to provide a diagnosis of the disease or to generate a treatment plan.

The healthcare portal may list a report for a patient if a given patient's acquired signal data set meets the signal quality standard. The report may indicate a disease-specific result (e.g., HCM) being available if the signal analysis could be performed. The patient's estimated score for the disease-specific analysis may be interpreted relative to an established threshold.

The report may be presented in the healthcare portal, e.g., to be used by a physician or healthcare provider in their diagnosis for indications of HCM. The indications include, in some embodiments, a probability or a severity score for the presence of a disease, medical condition, or an indication of either.

Outlier Assessment and Rejection Detection (538). Following the AE add-on module 514 computing the feature value outputs (in process 532) and prior to their application to the classifier models (in process 534), the AE add-on module 514 is configured in some embodiments to perform outlier analysis (shown in process 538) of the feature value outputs. Outlier analysis evaluation process 538 executes a machine-learned outlier detection module (ODM), in some embodiments, to identify and exclude anomalous acquired biophysical signals by identifying and excluding anomalous feature output values in reference to the feature values generated from the validation and training data. The outlier detection module assesses for outliers that present themselves within sparse clusters at isolated regions that are out of distribution from the rest of the observations. Process 538 can reduce the risk that outlier signals are inappropriately applied to classifier models and produce inaccurate evaluations to be viewed by the patient or healthcare provider. The accuracy of the outlier module has been verified using hold-out validation sets in which the ODM is able to identify all the labeled outliers in a test set with the acceptable outlier detection rate (ODR) generalization.

Experimental Results and Examples

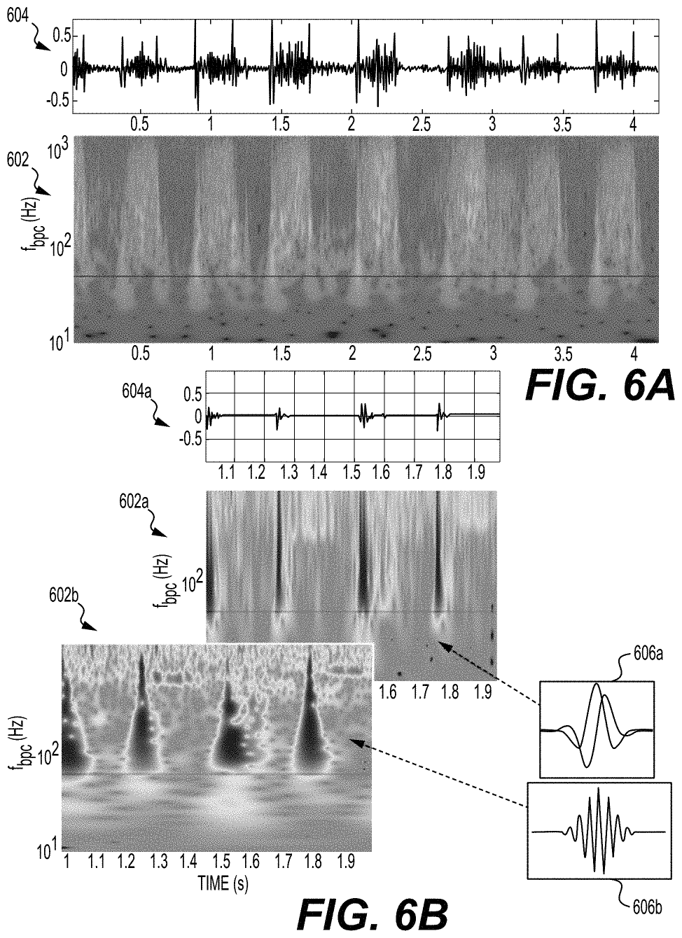

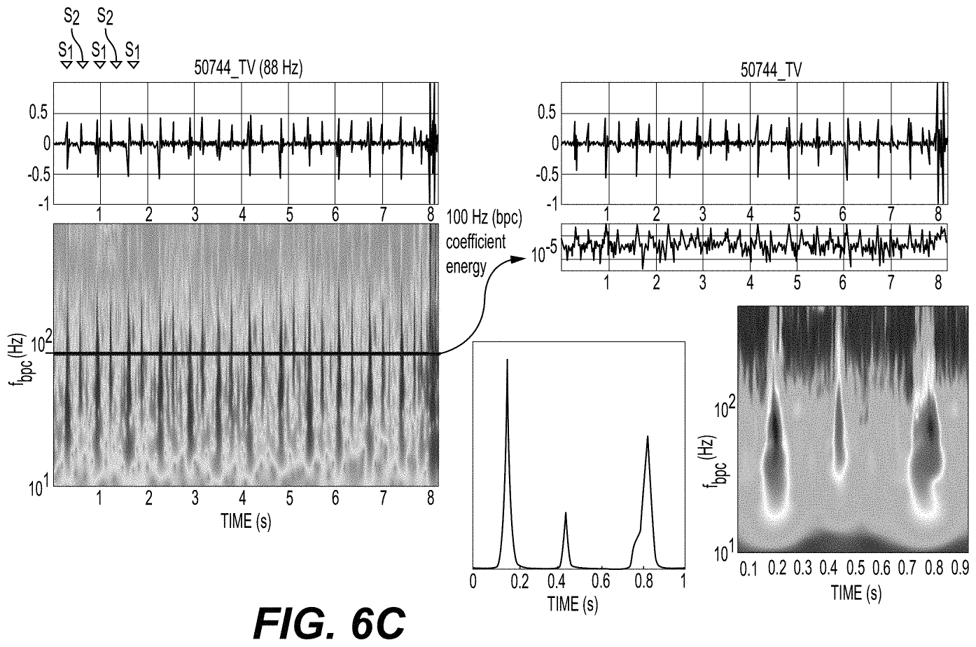

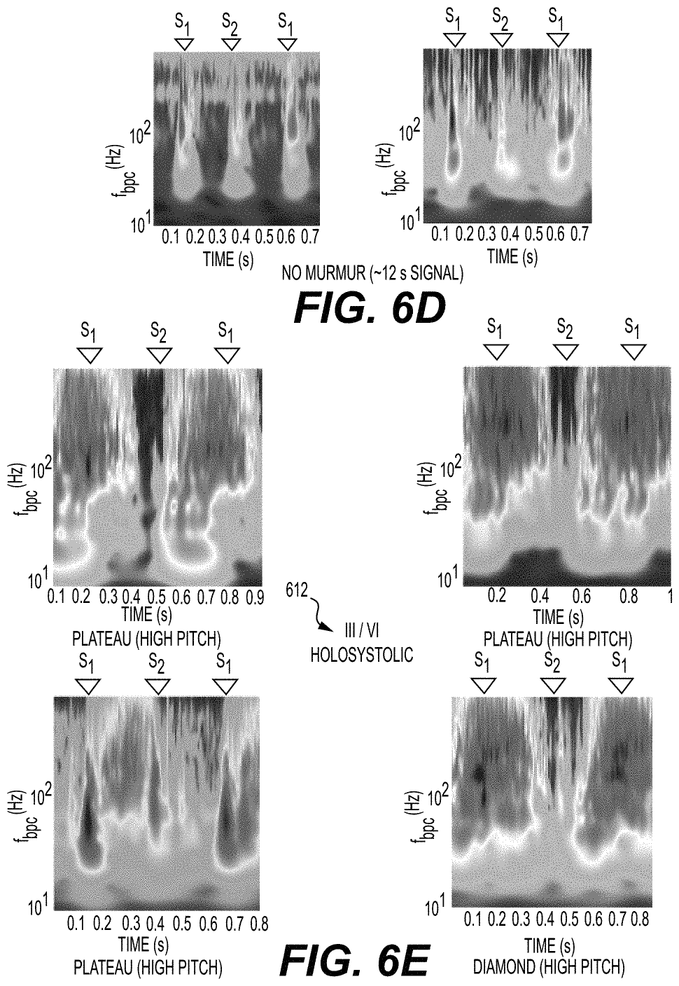

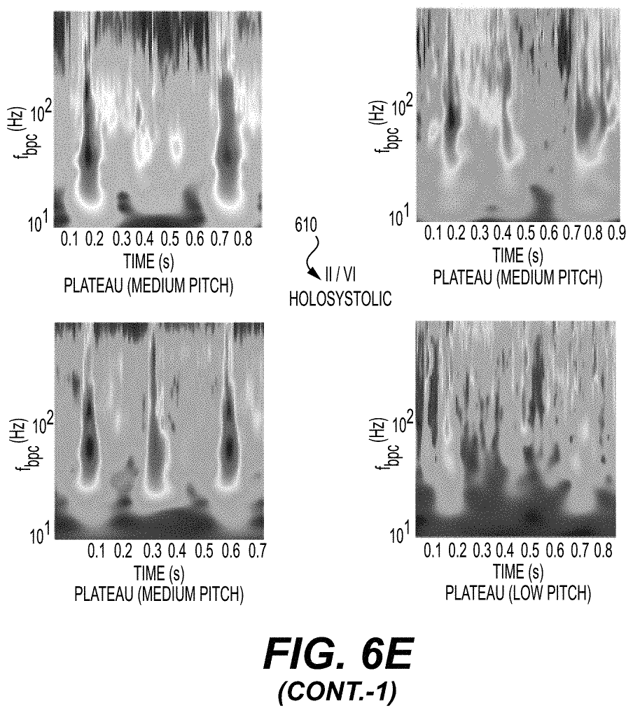

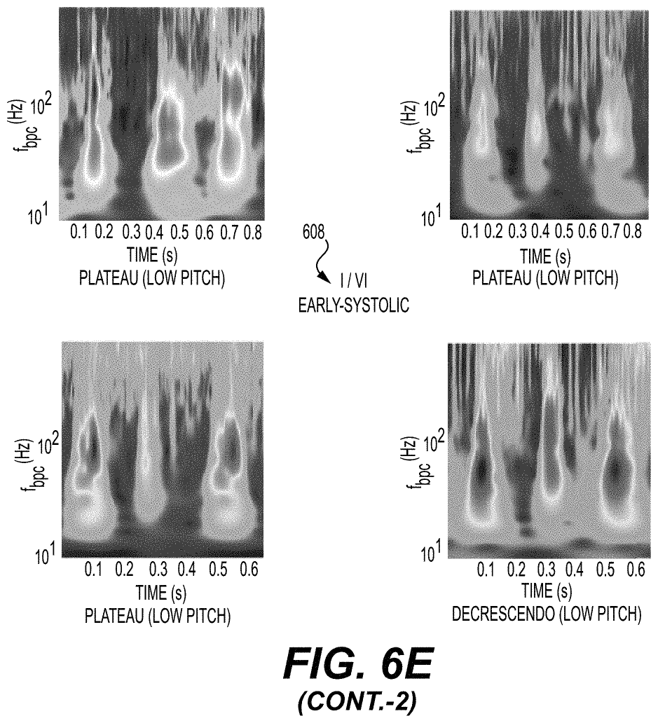

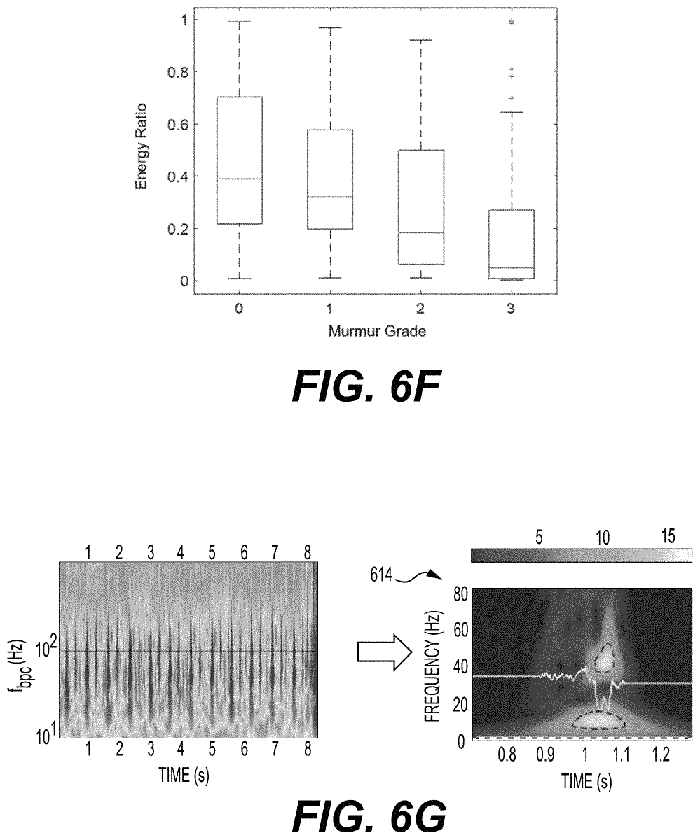

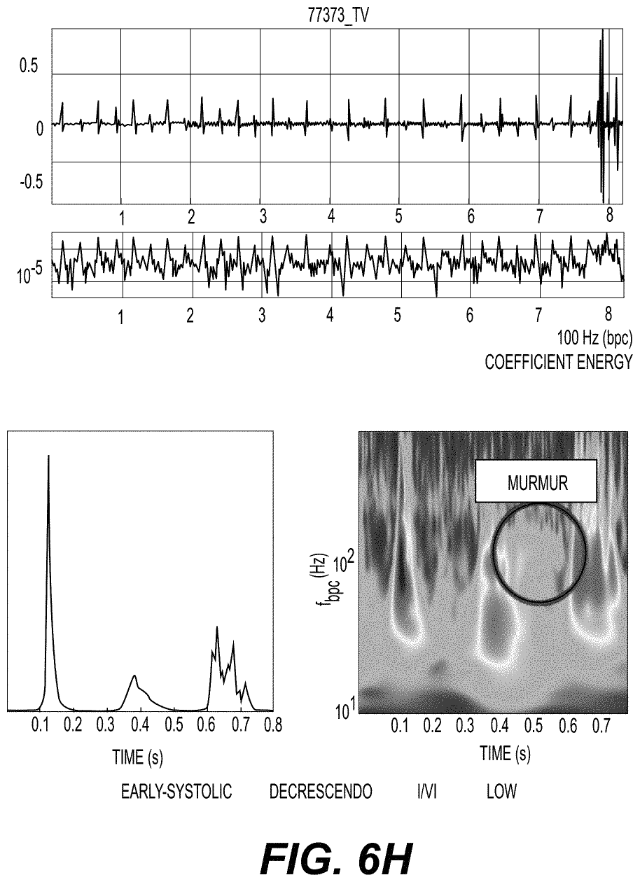

A study was conducted to evaluate ML features for the evaluation of phonocardiograms, e.g., to determine the presence or non-presence of HCM. FIGS. 6A-6G show experimental results of various developed ML features.

In FIG. 6A, a phonocardiographic/phonocardiogram (PCG) continuous wavelet transform (CWT) scalogram 602 is shown for a high-intensity systolic murmur generated from the DigiScope Phonocardiogram Dataset provided by Physionet. Similar signals may be acquired as acoustic signal 604, e.g., as acquired using the system of FIGS. 1 and 4A-4E. It can be observed from FIG. 6A that the holosystolic murmurs shown in scalogram 602 are high-pitched, i.e., having energy concentrated more at high frequencies.

FIG. 6B shows scalograms 602 (shown as 602a, 602b) with wavelet transforms 606 (shown as 606a, 606b) applied to the input signal 604 (shown as 604a). In FIG. 6B, a Morlet wavelet transform was applied. The transform 606a applies a lower co, for better resolution in the time domain, and transform 606b applies a higher co, for better resolution in the frequency domain. The wavelet may be configured by shape and by the mother wavelet and transform type.

FIG. 6C shows the results of an example operation to ensemble average the input signal 602 for both time and the CWT scalogram. In the operation, an energy scalogram is first calculated from the raw PCG. The operation then determines the maximum energy scales (e.g., −100 Hz) to identify the “S1” sounds using turning points and expected pulse rates. The “S1” fiducials (see, e.g., FIGS. 6D-6E) are then used as trigger points for the ensemble average derivations. In the results shown in FIG. 6C, a Morlet transform was performed with ω₀=5.5.

In some embodiments, a 1-D continuous wavelet transform is applied, such as a Morlet (also referred to as a Gabor) wavelet as the mother wavelet, as shown above. Other wavelets, e.g., having equal variance in time and frequency, may be used, e.g., Gaussian, Mexican Hat, Spline, and Mayer wavelet, etc. The wavelet may have a resolution of, e.g., 48 voices per octave. The Morlet wavelet is a wavelet composed of a complex exponential (carrier) multiplied by a Gaussian window (envelope), as shown in Equation 1.

ψ(t)=exp(iωt)exp(−t²/2σ²) (Equation 1)

In Equation 1, ω is the wavelet central frequency, and σ=n/2πf is the width of the Gaussian window with n (the number of cycles) controlling the time-frequency resolution trade-off.

Coherence waveforms may be determined as a measure of the correlation between time series signals, such as between the two observations of the phonocardiogram signal data set or between the phonocardiogram signal and the cardiac and/or photoplethysmographic signal data set(s). Wavelet coherence may be determined, for example, by Equation 2.

$${C}_{w} = \frac{{C}_{xy} ( a , τ )}{{\vert S ( {C}_{x} ( a , τ ) \vert}^{2} \cdot {\vert S ( {C}_{y} ( a , τ ) \vert}^{2}} \qquad ( Equation 2 )$$

In Equation 2, the cross-spectrum C_{xy} is a measure of the distribution of power of two signals x and y in the time-frequency domain given by Equation 3.

C_{xy}(a,τ)=|S(C_{xy}′(a,τ))|² (Equation 3)

In Equation 3, superscript * denotes a complex conjugate, and S is a smoothing operator in time and scale. In some embodiments, a coherence spectrum operator is performed, e.g., with 32 voices per octave, to find the coherence spectrum of the paired channels (e.g., between channels X and Y, channels x and X, and channels Y and Z). Coherence spectrum may be generated between (i) a phonocardiogram signal and (ii) the cardiac signal and/or a photoplethysmographic signal.

High-power spectral images or data may be generated from the scalogram, e.g., by generating a binarized spectral image of the spectral image or spectral data of the high-power spectral content of a waveform signal of interest, e.g., the spectral content of murmurs. FIG. 6G shows an example method of generating the binarized spectral image 614 of the spectral image or spectral data. The method may include performing a wavelet transformation to generate a spectral image or spectral data of a given waveform region of interest to which a threshold operator is applied to generate the binarized spectral image or data corresponding to the high spectral power characteristics of the waveform region. The computation may be performed over multiple heart cycles to extract, as wavelet-based features or parameters, a statistical characterization of the time range, frequency range, time centroid, surface area, eccentricity, circularity, extent, orientation, and/or power centroid of the high spectral energy characteristics of a waveform region. FIG. 6G shows an example of a binarized spectral image 614 generated from the spectral image or spectral data of the high-power spectral content of a waveform signal of interest from a scalogram of an input signal.

Examples of methods to generate such a binarized spectral image are provided in U.S. patent application Ser. No. 17/891,259, filed Aug. 18, 2022, and entitled “METHOD AND SYSTEM TO ASSESS DISEASE USING WAVELET ANALYSIS OF CARDIAC AND PHOTOPLETHYSMOGRAPHIC SIGNALS,” which is hereby incorporated by reference herein in its entirety.

Table 2 shows an example set of extractable high spectral energy characteristics of a waveform region of interest from a generated binarized spectral image or data.

TABLE 2

| Feature Name | Feature Description |
|---|---|
| Time range | The time duration of a high spectral energy region of the waveform of |
| | interest in a biophysical signal, the time duration, e.g., corresponding to |
| | a length (e.g., of the x-dimension) of a bounding box generated around |
| | a thresholded object corresponding to the high spectral energy region in |
| | a binarized spectral image or data. |
| Frequency range | The frequency range of a high spectral energy region of the waveform |
| | of interest in a biophysical signal, the frequency range, e.g., |
| | corresponding to the height (e.g., of the y-dimension) of a bounding box |
| | generated around a thresholded object corresponding to the high |
| | spectral energy region in the binarized spectral image or data. |
| Time centroid | The center of mass of the high spectral energy region of the waveform |
| | of interest in a biophysical signal, the center of mass determined from |
| | a binarized thresholded region in the time dimension (e.g., x-axis). |
| Frequency centroid | The center of mass of the high spectral energy region of the waveform |
| | of interest in a biophysical signal, the center of mass determined from |
| | a binarized thresholded region in the frequency dimension (e.g., y-axis). |
| Surface area | The size of the high spectral energy region of the waveform of interest |
| | in a biophysical signal, the size determined from a binarized |
| | thresholded region (e.g., in pixel) of the binarized spectral image. |
| Eccentricity | The eccentricity of the shape of the high spectral energy region of the |
| | waveform of interest in a biophysical signal, the eccentricity is |
| | determined as a ratio of the distance between (i) the foci of a fitted |
| | ellipse enclosing the binarized region and (ii) its major axis length (e.g., |
| | having a value between 0 and 1). An ellipse having an eccentricity of |
| | 0 is a circle, while an ellipse whose eccentricity is 1 is a line segment. |
| Circularity | The circularity of the shape of the high spectral energy region of the |
| | waveform of interest in a biophysical signal, the circularity determined |
| | $as \frac{4 \times surfaceArea \times π}{{Perimeter}^{2}} . For a circle , the circularity has a value of 1.$ |
| Extent | The extent of the shape of the high spectral energy region of the |
| | waveform of interest in a biophysical signal, the extent determined as a |
| | ratio of pixels in the binarized thresholded region to a number of pixels |
| | in a bounding box. |
| Orientation | The orientation of the shape of the high spectral energy region of the |
| | waveform of interest in a biophysical signal, the orientation being |
| | determined as an angle between the x-axis and the major axis of a fitted |
| | ellipse encompassing the binarized region (e.g., ranged from −90° to |
| | 90°). |
| Power centroid | The center of mass of the high spectral energy region of the waveform |
| | of interest in a biophysical signal, the power centroid being determined |
| | from a binarized thresholded region in the power dimension (e.g., z- |
| | axis or color). |
| NumRegions | The number of high spectral energy regions of the waveform of interest |
| | in a biophysical signal as determined from a number of binarized |
| | thresholded regions. |

In FIGS. 6D and 6E, it can be observed that the ensemble average output, as described in relation to FIG. 6C, can readily allow for the identification of pronounced murmurs (II-IV) in clean, well-behaved signals. FIG. 6D shows the ensemble average outputs for an acoustic signal with no murmur. FIG. 6E shows the ensemble average outputs for a set of acoustic signals with various stages of holosystolic (shown as “I/VI early-systolic” 608, “II/VI Holosystolic” 610, and “III/VI Holosystolic” 612). It can be observed that “pitch” appears to be correlated as much with the amplitude as with the frequency content. It can also be observed that a much higher proportion of grade “I” murmurs seem to be early-systolic rather than holosystolic.

FIG. 6F shows an example parametrization of the analysis of ensemble average output analysis of FIGS. 6D and 6E, e.g., per the features described in Table 1 and/or Table 2. The features of the parametrization may be employed in the classification output of the presence, non-presence, and/or severity of HCM, among other diseases and conditions described herein.

In some embodiments, the analysis may be performed in view of the respiration state of the patient. Examples of respiration estimation may be found in U.S. patent application Ser. No. 17/891,224, entitled “METHOD AND SYSTEM TO ASSESS DISEASE USING ESTIMATED RESPIRATION PARAMETERS FROM CARDIAC AND PHOTOPLETHYS-MOGRAPHIC SIGNALS.”

FIG. 6H shows an example scalogram having a detected murmur therein. The scalogram may be evaluated for the murmur using the wavelet feature analysis described herein.

Conclusion. While the methods and systems have been described in connection with certain embodiments and specific examples, it is not intended that the scope be limited to the embodiments set forth, as the embodiments herein are intended in all respects to be illustrative rather than restrictive. The clinical evaluation system and method discussed herein may be employed to make, or to assist a physician or other healthcare provider in making, noninvasive diagnoses or determinations of the presence, non-presence, and/or severity of other diseases and/or conditions, such as, e.g., coronary artery disease (CAD), pulmonary hypertension and other pathologies as described herein using similar or other development approach. In addition, the example clinical evaluation system and method can be used in the diagnosis and treatment of other cardiac-related pathologies and conditions as well as neurological-related pathologies and conditions; such assessment can be applied to the diagnosis and treatment (including surgical, minimally invasive, and/or pharmacologic treatment) of any pathologies or conditions in which a biophysical signal is involved in any relevant system of a living body. One example in the cardiac context is the diagnosis of CAD and other diseases and conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, conditions and/or diseases. Non-limiting examples of neurological-related diseases, pathologies or conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/conditions and vision-related diseases/conditions.

In addition, the clinical evaluation system described herein may be configured to analyze biophysical signals such as an electrocardiogram (ECG), electroencephalogram (EEG), gamma synchrony features in signals, respiratory function signals, photoplethysmographic, phonocardiographic, pulse oximetry signals, perfusion data signals; quasi-periodic biological signals, fetal ECG signals, blood pressure signals; cardiac magnetic field signals, heart rate signals, among others. As described herein, the clinical evaluation system may employ a single type of biophysical signal for HCM estimation, or it may employ multiple types of signals for HCM estimation. In addition, while it is contemplated, a 3-sensor system may be employed in which the third sensor is MEMS-based, (a) the third sensor could be non-MEMS-based, (b) it could be a single type of sensor (PPG, ECG, MEMS, etc.), (c) it could be a dual-sensor system (using three or more of two types of sensors), and (d) it could involve more than three sensors. Any combination of known sensor types, contact or non-contact (e.g., a non-contact thermometer) sensor, could be used.

Further examples of processing that may be used with the exemplified method and system disclosed herein are described in U.S. Pat. Nos. 9,289,150; 9,655,536; 9,968,275; 8,923,958; 9,408,543; 9,955,883; 9,737,229; 10,039,468; 9,597,021; 9,968,265; 9,910,964;

10,672,518; 10,566,091; 10,566,092; 10,542,897; 10,362,950; 10,292,596; 10,806,349; 11,133,109; 11,141,114; 11,160,509; 11,147,516; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. Design Patent Nos. D810947; D855064; D895661; D843382; D880501; D858532; U.S. patent application Ser. Nos. 16/232,586; 16/831,264; 16/429,593; 16/725,402; 16/831,380; 16/725,430; 16/725,416; 17/132,869; PCT Application Nos. PCT/IB2020/052889; PCT/IB2020/052890, each of which is hereby incorporated by reference herein in its entirety.

Each of the following patents, applications, and publications as listed below and throughout this document is hereby incorporated by reference herein in its entirety:

### LIST OF REFERENCES

[1] U.S. Provisional Patent Application No. 63/236,072, filed Aug. 23, 2021, entitled “METHOD AND SYSTEM TO ASSESS DISEASE USING VISUAL FEATURES OF CARDIAC AND PHOTOPLETHYSMOGRAPHIC SIGNALS,” having attorney docket No. 10321-49pv1.
[2] U.S. Provisional Patent Application No. 63/235,963, filed Aug. 23, 2021, entitled “METHOD AND SYSTEM TO ASSESS DISEASE USING POWER SPECTRAL ANALYSIS OF CARDIAC AND PHOTOPLETHYSMOGRAPHIC SIGNALS,” having attorney docket No. 10321-50pv1.
[3] U.S. Provisional Patent Application No. 63/235,968, filed Aug. 23, 2021, entitled “METHOD AND SYSTEM TO ASSESS DISEASE USING ESTIMATED RESPIRATION PARAMETERS FROM CARDIAC AND PHOTOPLETHYSMOGRAPHIC SIGNALS,” having attorney docket no. 10321-51pv1.
[4] U.S. Provisional Patent Application No. 63/235,968, filed Aug. 23, 2021, entitled “METHOD AND SYSTEM TO ASSESS DISEASE USING WAVELET ANALYSIS OF CARDIAC AND PHOTOPLETHYSMOGRAPHIC SIGNALS,” having attorney docket no. 10321-52pv1.
[5] U.S. patent application Ser. No. 17/558,702, filed Dec. 22, 2021, entitled “METHOD AND SYSTEM TO ASSESS DISEASE USING CYCLE VARIABILITY ANALYSIS OF BIOPHYSICAL SIGNALS,” having attorney docket no. 10321-053us1.
[6] U.S. Provisional Patent Application No. 63/235,971, filed Aug. 23, 2021, entitled “METHOD AND SYSTEM TO ASSESS DISEASE USING ANALYSIS OF PHOTOPLETHYSMOGRAPHIC SIGNALS,” having attorney docket no. 10321-054pv1.
[7] U.S. Provisional Patent Application No. 63/236,193, filed Aug. 23, 2021, entitled “METHOD AND SYSTEM TO ASSESS DISEASE USING MORPHOLOGICAL ANALYSIS OF CARDIAC SIGNALS,” having attorney docket no. 10321-055pv1.
[8] U.S. Provisional Patent Application No. 63/235,974, filed Aug. 23, 2021, entitled “METHOD AND SYSTEM TO ASSESS DISEASE USING DEPLORIZATION WAVE PROPAGATION DEVIATIONS,” having attorney docket no. 10321-056pv1.
[9] H. Zou and T. Hastie, “Regularization and variable selection via the elastic net,” Journal of the Roy. Statist. Soc., ser. B, vol. 67, no. 2, pp. 301-320, 2005.
[10] Breiman, “Random Forests,” Machine Learning, 45(1), 5-32, 2001.
[11] Chen, Tianqi; Guestrin, Carlos (2016). “XGBoost: A Scalable Tree Boosting System” In Krishnapuram, Balaji; Shah, Mohak; Smola, Alexander J.; Aggarwal, Charu C.; Shen, Dou; Rastogi, Rajeev (eds.). Proceedings of the 2²ⁿd ACM SIGKDD International Conference on Knowledge Discovery and Data Mining, San Francisco, Calif., USA, Aug. 13-17, 2016. ACM. pp. 785-794.
[12] U.S. patent application Ser. No. 16/831,264, filed Apr. 30, 2020, entitled “METHOD AND SYSTEM TO ASSESS DISEASE USING DYNAMICAL ANALYSIS OF BIOPHYSICAL SIGNALS,” having attorney docket no. 10321-040us1.
[13] U.S. patent application Ser. No. 16/831,380, filed Apr. 30, 2020, entitled “METHOD AND SYSTEM TO ASSESS DISEASE USING DYNAMICAL ANALYSIS OF CARDIAC AND PHOTOPLETHYSMOGRAPHIC SIGNALS,” having attorney docket no. 10321-041us1.
[14] U.S. application Ser. No. 17/486,609, filed Sep. 27, 2021, entitled “METHOD AND SYSTEM TO ASSESS DISEASE USING MULTI-SENSOR SIGNALS,” having attorney docket no. 10321-047 us1.
Fábio Leitão, Eurico Moreira, Filipe Alves, Mario Lourenço, Olga Azevedo, Joao Gaspar, and Luis A. Rocha, “High-Resolution Seismocardiogram Acquisition and Analysis System,” Journal of Sensors 2018, 18, 3441; doi:10.3390/s18103441.
[16] Martin S Maron, Jennifer L Hellawell, Jaime C Lucove, Ramin Farzaneh-Far, lacopo Olivotto, “Occurrence of Clinically Diagnosed Hypertrophic Cardiomyopathy in the United States,” Am J Cardiol 2016 May 15; 117(10):1651-1654.
[17] European heart journal cardiovascular imaging, 2018-10-01, Vol. 19 (10), p. 1126-1133
[18] U.S. Provisional Application No. 63/234,772, filed Aug. 19, 2021, entitled “MEDICAL EVALUATION SYSTEMS AND METHODS USING ADD-ON MODULES,” having attorney docket no. 10321-060pv1.
[19] U.S. Provisional Application No. 63/235,960, filed Aug. 23, 2021, entitled “METHOD AND SYSTEM TO NON-INVASIVELY ASSESS ELEVATED LEFT VENTRICULAR END-DIASTOLIC PRESSURE,” having attorney docket no. 10321-048pv1.
[20] https://www.umms.org/ummc/health-services/heart-vascular/services/hypertrophic-cardiomyopathy/types-symptoms-causes
[21] Raj M A, Ranka S, Goyal A., “Hypertrophic Obstructive Cardiomyopathy,” [Updated 2021 Nov. 4]. In: StatPearls [Internet]. Treasure Island (FL): StatPearls Publishing; 2022 January. Available from: https://www.ncbi.nlm.nih.gov/books/NBK430820/
[21] Maron B J, Wolfson J K, Roberts W C. Relation between extent of cardiac muscle cell disorganization and left ventricular wall thickness in hypertrophic cardiomyopathy. Am J Cardiol. 1992 Sep. 15; 70(7):785-90.
[22] Marian A J, Braunwald E. Hypertrophic Cardiomyopathy: Genetics, Pathogenesis, Clinical Manifestations, Diagnosis, and Therapy. Circ Res. 2017; 121(7):749-770. doi:10.1161/CIRCRESAHA.117.311059.
[23] Electrophysiologic abnormalities in patients with hypertrophic cardiomyopathy. A consecutive analysis in 155 patients, L Fananapazir, C M Tracy, M B Leon, J B Winkler, R O Cannon, ^{3r}d, R O Bonow, B J Maron, and S E Epstein.
[24] Dubin, Dale. Rapid Interpretation of EK's: An Interactive Course. Sixth edition. Tampa, Fla.: Cover Pub. Co., 2000.
[25] The electrocardiogram in the diagnosis and management of patients with hypertrophic cardiomyopathy, Finocchiaro, Gherardo, et al., Heart Rhythm, Volume 17, Issue 1, 142-151.
[26] Savelieva, Irina, et al.“Comparative reproducibility of QT, QT peak, and T peak-T end intervals and dispersion in normal subjects, patients with myocardial infarction, and patients with hypertrophic cardiomyopathy” Pacing and clinical electrophysiology 21.11 (1998): 2376-2381.
[27] Dinshaw, Leon, et al. “The T-peak-to-T-end interval: a novel ECG marker for ventricular arrhythmia and appropriate ICD therapy in patients with hypertrophic cardiomyopathy,” Clinical Research in Cardiology 107.2 (2018): 130-137.
[28] Finocchiaro, Gherardo, et al. “The electrocardiogram in the diagnosis and management of patients with hypertrophic cardiomyopathy” Heart Rhythm 17.1 (2020): 142-151.
[29] Jothiramalingam, R., Jude, A., Patan, R. et al. Machine learning-based left ventricular hypertrophy detection using multi-lead ECG signal. Neural Comput & Applic 33, 4445-4455 (2021). https://doi.org/10.1007/s00521-020-05238-2.
[30] A. Englund, K. Hnatkova, P. M. Elliot, W. J. McKenna, M. Malik, Wavelet decomposition analysis of the signal averaged electrocardiogram used for risk stratification of patients with hypertrophic cardiomyopathy, European Heart Journal, Volume 19, Issue 9, 1 Sep. 1998, Pages 1383-1390,
[31] Edhouse J, Thakur R K, Khalil J M. ABC of clinical electrocardiography. Conditions affecting the left side of the heart. BMJ. 2002 May 25; 324(7348):1264-7.
[32] Harrigan R A, Jones K. ABC of clinical electrocardiography. Conditions affecting the right side of the heart. BMJ. 2002 May 18; 324(7347):1201-4.
[33] Green, E. M., van Mourik, R., Wolfus, C. et al. Machine learning detection of obstructive hypertrophic cardiomyopathy using a wearable biosensor. npj Digit. Med. 2, 57 (2019).
[34] Eleid M F, Konecny T, Orban M, Sengupta P P, Somers V K, Parish J M, Mookadam F, Brady P A, Sullivan B L, Khandheria B K, Ommen S R, Tajik A J., “High prevalence of abnormal nocturnal oximetry in patients with hypertrophic cardiomyopathy,” J Am Coll Cardiol. 2009 Nov. 3; 54(19):1805-9.
[35] Andrés Ricardo Pérez-Riera, “Electro/Vectorcardiogram in Left Ventricular Hypertrophy/Enlargement-Part 1,” retrieved on 13 Jan. 2022 from http://cardiolatina.com/wp-content/uploads/2020/07/ECG-VCG-in-Left-Ventricular-Hypertrophy-Enlargements-LVH-Part-1.pdf.
[36] Erick Jimenez, Amr El-Bokl, Daniel Cortez, “Vectorcardiography as a prognostic tool in hypertrophic cardiomyopathy,” Journal of Electrocardiology, Volume 68, 2021, Pages 80-84, ISSN 0022-0736.
[37] Pérez-Riera A R, de Lucca A A, Barbosa-Barros R, et al., “Value of electro-vectorcardiogram in hypertrophic cardiomyopathy,” Ann. Noninvasive Electrocardiol. 2013; 18(4):311-326.
[38] Green, E. M., van Mourik, R., Wolfus, C. et al., “Machine learning detection of obstructive hypertrophic cardiomyopathy using a wearable biosensor,” npj Digit. Med. 2, 57 (2019).
[39] Houston B A, Stevens G R, “Hypertrophic cardiomyopathy: a review,” Clin Med Insights Cardiol. 2015; 8 (Suppl 1):53-65. Published 2015 Jan. 26. doi:10.4137/CMC.S15717.
[40] Takahashi K, Ono K, Arai H, et al., “Detection of Pathologic Heart Murmurs Using a Piezoelectric Sensor,” Sensors (Basel). 2021; 21(4):1376. Published 2021 Feb. 16. doi:10.3390/s21041376.
[41] Folino, A. F., Buja, G., Bauce, B., Thiene, G., Volta, S. D. And Nava, A., “Heart Rate Variability in Arrhythmogenic Right Ventricular Cardiomyopathy Correlation with Clinical and Prognostic Features,” Pacing and Clinical Electrophysiology, 25: 1285-1292 (2002).
[42] F. Laurent et al., “Heart Rate Variability in Idiopathic Dilated Cardiomyopathy: Characteristics and Prognostic Value,” Journal of the American College of Cardiology, 1997; 30(4):1109-1014.
[43] Tavakolian, Kouhyar, “Systolic time intervals and new measurement methods,” Cardiovascular engineering and technology 7.2 (2016): 118-125.
[44] Reant P, Dijos M, Donal E, Mignot A, Ritter P, Bordachar P, Dos Santos P, Leclercq C, Roudaut R, Habib G, Lafitte S, “Systolic time intervals as simple echocardiographic parameters of left ventricular systolic performance: correlation with ejection fraction and longitudinal two-dimensional strain,” Eur J Echocardiogr. 2010 December; 11(10):834-44.
[45] Claria, Francesc, et al., “Heart rate variability analysis based on time—frequency representation and entropies in hypertrophic cardiomyopathy patients,” Physiological measurement 29.3 (2008): 401.
[46] U.S. patent application Ser. No. 17/891,526, entitled “METHOD AND SYSTEM TO ASSESS DISEASE USING VISUAL FEATURES OF CARDIAC AND PHOTOPLETHYSMOGRAPHIC SIGNALS.”
[47] U.S. patent application Ser. No. 17/891,229, entitled “METHOD AND SYSTEM TO ASSESS DISEASE USING POWER SPECTRAL ANALYSIS OF CARDIAC AND PHOTOPLETHYSMOGRAPHIC SIGNALS.”
[48] U.S. patent application Ser. No. 17/891,380, entitled “METHOD AND SYSTEM TO ASSESS DISEASE USING DEPLORIZATION WAVE PROPAGATION DEVIATIONS.”
[49]U.S. patent application Ser. No. 17/558,702, entitled “METHOD AND SYSTEM TO ASSESS DISEASE USING CYCLE VARIABILITY ANALYSIS OF BIOPHYSICAL SIGNALS.”
[50] U.S. Pat. No. 11,291,379.
[51] U.S. Publication No. 2020/0397322.
[52] U.S. patent application Ser. No. 17/891,278, entitled “METHOD AND SYSTEM TO ASSESS DISEASE USING ANALYSIS OF PHOTOPLETHYSMOGRAPHIC SIGNALS.”
[53] U.S. patent application Ser. No. 17/891,259, entitled “METHOD AND SYSTEM TO ASSESS DISEASE USING WAVELET ANALYSIS OF CARDIAC AND PHOTOPLETHYSMOGRAPHIC SIGNALS.”
[54] U.S. patent application Ser. No. 17/891,526, entitled “METHOD AND SYSTEM TO ASSESS DISEASE USING VISUAL FEATURES OF CARDIAC AND PHOTOPLETHYSMOGRAPHIC SIGNALS.”
[55] U.S. patent application Ser. No. 17/891,224, entitled “METHOD AND SYSTEM TO ASSESS DISEASE USING ESTIMATED RESPIRATION PARAMETERS FROM CARDIAC AND PHOTOPLETHYSMOGRAPHIC SIGNALS.”
[56] U.S. patent application Ser. No. 17/891,533, entitled “METHOD AND SYSTEM TO ASSESS DISEASE USING MORPHOLOGICAL ANALYSIS OF CARDIAC SIGNALS.”

1. (canceled)

2. A method to non-invasively estimate a presence, non-presence, and/or severity of hypertrophic cardiomyopathy in a mammalian subject, the method comprising:
obtaining, by one or more processors, one or more seismocardiographic signals (SCG signals) and/or phonocardiographic signals (PCG signals) from a multi-sensor device placed or worn on a patient;
determining, by the one or more processors utilizing at least a portion of the one or more seismocardiographic signals and/or phonocardiographic signals, a plurality of values associated with a plurality of features or machine-learned-based analyses; and
determining, by the one or more processors, an estimated value for the presence, non-presence and/or severity of hypertrophic cardiomyopathy using the plurality of values associated with the plurality of features or machine-learned-based analyses,
outputting, by the one or more processors, the estimated value for the presence, non-presence, and/or severity of hypertrophic cardiomyopathy, wherein the estimated value for the presence, non-presence, and/or severity of hypertrophic cardiomyopathy is outputted for use in a diagnosis of hypertrophic cardiomyopathy and/or to direct treatment of the hypertrophic cardiomyopathy.

3. (canceled)

4. The method of claim 2, wherein the plurality of features or machine-learned-based analyses are configured to quantify deviations of a VD wave trajectory from a trajectory of a three-dimensional-modeled VD wave.

5. The method of claim 2, wherein the plurality of features or machine-learned-based analyses are configured to quantify beat-to-beat variations in cardiac signals.

6. The method of claim 2, wherein the plurality of features or machine-learned-based analyses are configured to quantify variability in registered landmarks in cardiac, PPG, SCG, and/or PCG signals via Poincare analysis and histogram analysis.

7. The method of claim 2, wherein the plurality of features or machine-learned-based analyses are configured to quantify dynamical characteristics of cardiac, PPG, SCG, and/or PCG signals.

8. The method of claim 2, wherein the plurality of features or machine-learned-based analyses are configured to quantify (i) properties of cardiac, PPG, and/or SCG signals.

9. The method of claim 2, wherein the plurality of features or machine-learned-based analyses are configured to quantify main frequency components of a cardiac, PPG, SCG, and/or PCG signals signal using wavelet analysis.

10. The method of claim 2, wherein the plurality of features or machine-learned-based analyses are configured to quantify power spectrum and frequency contents of the SCG and/or PCG signals using power spectrum and coherence analysis.

11. The method of claim 2, wherein the plurality of features or machine-learned-based analyses are configured to quantify properties of the SCG and/or PCG signals over loop regions in 3D phase spaces, projections thereof, and loop vectors.

12. The method of claim 2, wherein the plurality of features or machine-learned-based analyses are configured to approximate a respiration waveform using either (i) PPG and cardiac signals or (ii) SCG and/or PCG signals to assess one of a (1) heart rate variability, (2) respiration rate, (3) discrepancy features representing a distance between respiration and modulation signals and (4) square coherence representing a correlation between modulation and respiration rate signals, wherein the approximated respiration waveform is employed for HCM assessment by being used to generate delineated inspiration and expiration portions of the SCG signals and/or PCG signals to be employed for the analysis.

13. The method of claim 2, wherein the plurality of features or machine-learned-based analyses are configured to quantify physiological aspects of the SCG and/or PCG signals.

14. The method of claim 2, wherein the plurality of features or machine-learned-based analyses are configured to quantify characteristic variations in SCG and/or PCG signals associated with inspiration versus expiration versus a Valsalva maneuver to identify patients with HCM.

15. The method of claim 2, wherein the plurality of features or machine-learned-based analyses are configured to quantify characteristic variations in SCG and/or PCG signals associated with inspiration versus expiration versus a Valsalva maneuver to identify a subset of patients with HCM that have obstructive HCM (OHCM).

16. The method of claim 2, wherein the plurality of features or machine-learned-based analyses are configured to approximate left ventricular ejection time using the one or more SCG and/or PCG signals.

17. The method of claim 2, wherein the plurality of features or machine-learned-based analyses are configured to quantify propagative characteristics of a ventricular depolarization (VD) wave and/or ventricular repolarization (VR) wave in three-dimensional space.

18. The method of claim 2, wherein the plurality of features or machine-learned-based analyses are evaluated (i) at an inspiration region of the one or more seismocardiographic signals and/or phonocardiographic signals and/or (ii) an expiration region of the one or more seismocardiographic signals and/or phonocardiographic signals.

19. (canceled)

20. An apparatus comprising:
a sensor body configured to be externally worn or placed on a chest region of a subject to acquire biophysical signals from the subject's chest region, including signals of the subject's heart; and
two or more MEMS-based sensors, including a first MEMS-based sensor and a second MEMS-based sensor, wherein the two or more MEMS-based sensors are located within the sensor body and connected to an electrode configured to be placed on a subject,
wherein the first MEMS-based sensor and the second MEMS-based sensor during operation generate a first seismographic signal and/or a first acoustic signal and a second seismographic signal and/or a second acoustic signal to be provided to an analysis system configured to evaluate a plurality of features or machine-learned-based analyses to generate an estimated value for a presence, non-presence, and/or severity of hypertrophic cardiomyopathy.

21. The apparatus of claim 19 further comprising:
a plurality of surface electrodes configured to be placed on surfaces of a chest region of a subject to provide a plurality of cardiac signals of the subject's heart,
wherein the plurality of cardiac signals are provided to the analysis system to evaluate for the plurality of features or machine-learned-based analyses to generate the estimated value for the presence, non-presence, and/or severity of hypertrophic cardiomyopathy; and
a plurality of photoplethysmographic sensors configured to be placed on the subject to provide one or more photoplethysmographic signals,
wherein the one or more photoplethysmographic signals are provided to the analysis system to evaluate for the plurality of features or machine-learned-based analyses to generate the estimated value for the presence, non-presence, and/or severity of hypertrophic cardiomyopathy.

22. (canceled)

23. The apparatus of claim 20, wherein the first MEMS-based sensor as an accelerometer or an acoustic sensor is configured to be placed non-invasively on the chest of the subject proximal to an apex region of the subject's heart.

24. The apparatus of claim 20, wherein the second MEMS-based sensor as an accelerometer or an acoustic sensor is configured to be placed non-invasively on the chest of the subject proximal to a base region of the subject's heart.

25. (canceled)

26. A non-transitory computer-readable medium comprising instructions stored thereon, wherein execution of the instructions by one or more processors causes the one or more processors to:
obtain one or more seismocardiographic signals (SCG signals) and/or phonocardiographic signals (PCG signals) from a multi-sensor device placed or worn on a patient;
determine utilizing at least a portion of the one or more seismocardiographic signals and/or phonocardiographic signals, a plurality of values associated with a plurality of features or machine-learned-based analyses;
determine an estimated value for a presence, non-presence and/or severity of hypertrophic cardiomyopathy using the plurality of values associated with the plurality of features or machine-learned-based analyses; and
output the estimated value for the presence, non-presence, and/or severity of hypertrophic cardiomyopathy, wherein the estimated value for the presence, non-presence, and/or severity of hypertrophic cardiomyopathy is outputted for use in a diagnosis of hypertrophic cardiomyopathy and/or to direct treatment of the hypertrophic cardiomyopathy.

27.-28. (canceled)
